(19) United States
(12) Patent Application Publication
(10) Pub. No.: US 2021/0393685 A1
(43) Pub. Date: Dec. 23, 2021

(54) ANTI-CD33 AND NKG2D LIGAND CHIMERAS FOR TREATMENT OF MYELOID MALIGNANCIES

(71) Applicant: THE TRUSTEES OF COLUMBIA UNIVERSITY IN THE CITY OF NEW YORK, New York, NY (US)

(72) Inventors: Siddhartha MUKHERJEE, New York, NY (US); Abdullah Mahmood ALI, New York, NY (US)

(21) Appl. No.: 17/182,422

(22) Filed: Feb. 23, 2021

Related U.S. Application Data
(63) Continuation of application No. PCT/US2019/048138, filed on Aug. 26, 2019.
(60) Provisional application No. 62/722,642, filed on Aug. 24, 2018.

(51) Int. Cl. A61K 35/17; C07K 14/735; C07K 14/705

(52) U.S. Cl. CPC A61K 35/17; C07K 14/70535; C07K 2319/95; C07K 2319/30; C07K 2319/33; C07K 14/70507

(57) ABSTRACT

Disclosed herein are compositions and methods relating to fusion proteins that target a lineage-specific cell-surface antigen for treating hematological malignancies.

### CROSS REFERENCE TO RELATED APPLICATIONS

This application is a continuation of PCT Application No. PCT/US19/48138, filed on Aug. 26, 2019, which claims priority to U.S. Provisional Application No. 62/722,642 filed on Aug. 24, 2018, all of which are incorporated by reference, as if expressly set forth in their respective entireties herein.

### INCORPORATION-BY-REFERENCE OF SEQUENCE LISTING

A sequence listing, filed as the ASCII text file “01001-006138-WO₀_ST25.txt” having a file size of 46 kilobytes, is incorporated herein by reference in its entirety.

### BACKGROUND

Despite decades of attempts, curative immunological therapy against cancer has been very difficult to achieve, with the fundamental basis being antigen-recognition capacity, either by antibodies or through T cells (via the T cell receptor) (Cousin-Frankel, Science (2013) 342:1432). Antibody-based immunotherapies have been used extensively against cancer in instances where the target antigen is up-regulated in tumor cells as compared to normal cells (e.g., Her-2 in Her-2 amplified breast cancer), or in cases where the tumor cells express an antigen that can be recognized by the antibody or an antibody-toxin conjugate (e.g., Rituximab against CD20) (Baselga et al., Annals Oncology (2001) 12:S35). While clinical trials using antibody-based immunotherapies have shown improved patient survival in a limited number of cancer types (usually when combined with standard chemotherapy), these effects are often accompanied by significant safety and efficacy concerns (Cousin-Frankel, Science (2013) 342:1432).

The role of the immune system in cancer is related to immunosurveillance—a process by which the immune system monitors the body for transformed cells in order to destroy them. Natural killer cells (NK cells) form one of the first lines of defense against pathogens and tumors (Biron, et al. (1999) Annu. Rev. Immunol. 17:189-220; Trinchieri (1989) Adv. Immunol. 47:187-376).

NK cells are a type of cytotoxic lymphocyte critical to the innate immune system. The role NK cells play is analogous to that of cytotoxic T cells in the vertebrate adaptive immune response. NK cells provide rapid responses to virus-infected cells, and respond to tumor formation. Typically, immune cells detect the major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. Innate effector cells, such as NK cells, recognize and eliminate their targets with fast kinetics, without prior sensitization. Therefore, NK cells can sense if cells are transformed, infected, or stressed to discriminate between abnormal and healthy tissues. This role is especially important because harmful cells that are missing MHC I markers cannot be detected and destroyed by other immune cells, such as T lymphocyte cells.

The role of NK cells in both the innate and adaptive immune responses is becoming increasingly important in research using NK cell activity as a potential cancer therapy. NK cells may contain both activating and inhibitory NK cell receptors which play important functional roles. Activating NK cell receptors specific for classic MHC class I molecules, nonclassic MHC class I molecules or MHC class I-related molecules have been described (Bakker, et al. (2000) Hum. Immunol. 61:18-27). One such receptor is NKG2D (natural killer cell group 2D) which is a C-type lectin-like receptor expressed on NK cells, gamma delta-TcR+ T cells, and CD8+ alpha beta-TcR+ T cells (Bauer, et al. (1999) Science 285:727-730). NKG2D is associated with the transmembrane adapter protein DAP10 (Wu, et al. (1999) Science 285:730-732), whose cytoplasmic domain binds to the p85 subunit of the PI-3 kinase. Tumor immune surveillance can be mediated by the NKG2D receptor, which stimulates natural killer (NK) and CD8 T cell responses against cancer cells expressing its ligands.

Currently, new approaches are needed for diseases such as acute myeloid leukemia (AML) in which the outcomes in older patients who are unable to receive intensive chemotherapy, the current standard of care, remains very poor, with a median survival of only 5 to 10 months (Dohner et al., NEJM (2015) 373:1136).

### SUMMARY

The present disclosure provides for a fusion polypeptide. The fusion polypeptide may comprise: (i) an antigen-binding fragment that binds a lineage-specific cell-surface antigen; and (ii) a polypeptide that binds a molecule expressed on natural killer (NK) cells.

The present disclosure provides for a composition comprising: (i) a first antigen-binding fragment that binds a lineage-specific cell-surface antigen; and (ii) a second polypeptide that binds a molecule expressed on natural killer (NK) cells, where the first antigen-binding fragment comprises a first dimerization motif, where the second polypeptide comprises a second dimerization motif, and where the first dimerization motif binds to the second dimerization motif.

The first dimerization motif and/or the second dimerization motif may comprise IgA, a leucine zipper motif, or an Fc region of an antibody.

The molecule expressed on NK cells may be a ligand or receptor expressed on NK cells. The molecule expressed on NK cells may be NKG2D, CD16, or CD2.

The polypeptide that binds the molecule expressed on NK cells (or the second polypeptide that binds a molecule expressed on NK cells) may be a ligand for a NK cell surface receptor.

The polypeptide that binds a molecule expressed on NK cells (or the second polypeptide that binds a molecule expressed on NK cells) may be ULBP1, ULBP2, ULBP3, ULBP4, ULBP5, ULBP6, MICA, MICB, or homologs, mutants or fragments thereof. In certain embodiments, the polypeptide that binds a molecule expressed on NK cells is an ectodomain of ULBP1, ULBP2, ULBP3, ULBP4, ULBP5, ULBP6, MICA, or MICB.

The lineage-specific cell-surface antigen may be CD33, CD19, or any of the lineage-specific cell-surface antigen described herein.

The antigen-binding fragment may be a single-chain antibody fragment (scFv).

In certain embodiments, the antigen-binding fragment comprises an amino acid sequence at least 80% or at least 90% identical to the amino acid sequence set forth in SEQ ID NO: 10.

In certain embodiments, the antigen-binding fragment comprises an amino acid sequence at least 80% or at least 90% identical to the amino acid sequence set forth in SEQ ID NO: 15.

In certain embodiments, the antigen-binding fragment comprises (i) an amino acid sequence at least 80% or at least 90% identical to the amino acid sequence set forth in SEQ ID NO: 10, and (ii) an amino acid sequence at least 80% or at least 90% identical to the amino acid sequence set forth in SEQ ID NO: 15.

In certain embodiments, the antigen-binding fragment comprises a heavy chain variable region comprising complementary determining regions (CDRs, e.g., CDR1, CDR2, CDR3) comprising amino acid sequences at least 80% or at least 90% identical to the amino acid sequences set forth in SEQ ID NO: 16, SEQ ID NO: 17, and SEQ ID NO: 18, respectively.

In certain embodiments, the antigen-binding fragment comprises a light chain variable region comprising complementary determining regions (CDRs, e.g., CDR1, CDR2, CDR3) comprising amino acid sequences at least 80% or at least 90% identical to the amino acid sequences set forth in SEQ ID NO: 11, SEQ ID NO: 12, and SEQ ID NO: 13, respectively.

In certain embodiments, the fusion polypeptide comprises an amino acid sequence at least 80% or at least 90% identical to the amino acid sequence set forth in SEQ ID NO: 20, SEQ ID NO: 21, or SEQ ID NO: 22.

The present disclosure provides for a composition comprising the present fusion polypeptide, or a nucleic acid molecule encoding the fusion polypeptide.

The present disclosure also provides for a nucleic acid molecule encoding a fusion polypeptide. The fusion polypeptide may comprise: (i) an antigen-binding fragment that binds a lineage-specific cell-surface antigen; and (ii) a polypeptide that binds a molecule expressed on natural killer (NK) cells.

The present disclosure provides for a vector comprising the present nucleic acid molecule, or a composition comprising the present nucleic acid molecule.

The present disclosure provides for a cell comprising the present vector or nucleic acid molecule.

The present disclosure provides for a composition comprising at least one vector encoding: (i) a first antigen-binding fragment that binds a lineage-specific cell-surface antigen; and (ii) a second polypeptide that binds a molecule expressed on natural killer (NK) cells, where the first antigen-binding fragment comprises a first dimerization motif, where the second polypeptide comprises a second dimerization motif, and where the first dimerization motif binds to the second dimerization motif.

The present disclosure provides for a composition comprising the present fusion polypeptide, the present nucleic acid molecule, the present vector, and/or the present cell.

Also encompassed by the present disclosure is a kit comprising the present fusion polypeptide, the present nucleic acid molecule, the present vector, the present cell, and/or the present composition.

The present disclosure provides for a method of treating a hematopoietic malignancy in a subject, comprising administering to the subject an effective amount of the present fusion polypeptide, the present nucleic acid molecule, the present vector, the present cell, and/or the present composition.

The hematopoietic malignancy may be a myeloid malignancy.

The hematopoietic malignancy may be Hodgkin's lymphoma, non-Hodgkin's lymphoma, leukemia, or multiple myeloma.

The hematopoietic malignancy may be acute myeloid leukemia, chronic myelogenous leukemia, acute lymphoblastic leukemia, or chronic lymphoblastic leukemia.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 presents an exemplary illustration of type 0, type 1, type 2, and type 3 lineage-specific antigens.

FIGS. 2A-2B present a scheme showing the design of the protein chimeras with anti-CD33 and ULBP1 (FIG. 2A), and the relevant sequences (FIG. 2B).

FIGS. 3A-3B present a scheme showing the design of the protein chimeras with ULBP1 and anti-CD33 (FIG. 3A), and the relevant sequences (FIG. 3B).

FIGS. 4A-4B present expression of anti-CD33-ULBP1 chimeras in cells (FIG. 4A), and expression and purification of anti-CD33-ULBP1 chimeras in supernatant (FIG. 4B). FIG. 4A: Lane 1: protein standard; Lanes 2 and 3: mock; Lanes 4 and 5: Chimera 1 (CD33-ULBP1-1 in FIGS. 2A-2B); Lanes 6 and 7: Chimera 2 (CD33-ULBP1-2 in FIGS. 2A-2B); Lanes 8 and 9: protein standard. FIG. 4B: Lane 1: protein standard; Lane 2: mock supernatant; Lane 3: mock flow-through; Lane 4: mock elute; Lane 5: Chimera 1 supernatant; Lane 6: Chimera 1 flow-through; Lane 7: Chimera 1 elute; Lane 8: Chimera 2 supernatant; Lane 9: Chimera 2 flow-through; Lane 10: Chimera 2 elute; Lanes 11 and 12: protein standard.

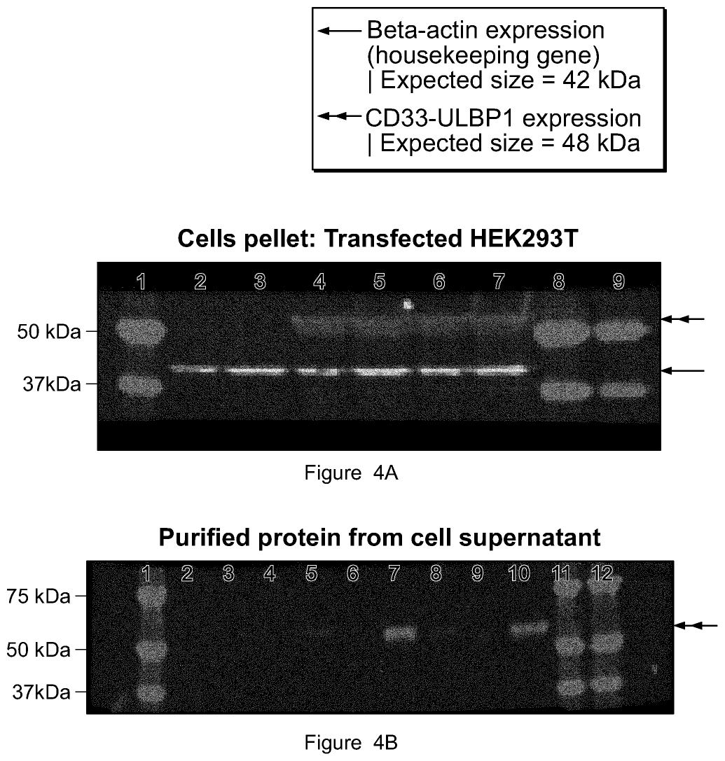

FIGS. 5A-5E are flow cytometry dot plots showing specific binding of fusion proteins with primary NK cells. FIG. 5A: NK cells only (control). FIG. 5B: NK cells+anti-Myc Ab (control). FIG. 5C: NK cells+mock+anti-Myc Ab (control). FIG. 5D: NK cells+Chimera 1+anti-Myc Ab. FIG. 5E: NK cells+Chimera 2+anti-Myc Ab.

FIGS. 6A-6E are flow cytometry dot plots showing specific binding of fusion proteins with CD34⁺ cells. FIG. 6A: CD34⁺ cells only (control). FIG. 6B: CD34⁺ cells+anti-Myc Ab (control). FIG. 6C: CD34⁺ cells+mock+anti-Myc Ab (control). FIG. 6D: CD34⁺ cells+Chimera 1+anti-Myc Ab. FIG. 6E: CD34⁺ cells+Chimera 2+anti-Myc Ab.

### DETAILED DESCRIPTION

The present disclosure provides for agents comprising an antigen-binding fragment that binds a lineage-specific cell-surface antigen (e.g., CD33) which can cause cell death of the cells expressing the lineage-specific cell-surface antigen. Immunotherapies involving the combination of an antigen-binding fragment that binds a lineage-specific cell-surface antigen (e.g., CD33), and a polypeptide that binds a molecule expressed on natural killer (NK) cells, would provide an efficacious method of treatment for hematopoietic malignancies.

The present disclosure provides for a fusion polypeptide, comprising (or consisting essentially of, or consisting of): (i) an antigen-binding fragment that binds a lineage-specific cell-surface antigen (e.g., CD33); and (ii) a polypeptide that binds a molecule expressed on natural killer (NK) cells.

The present disclosure provides for a fusion polypeptide, comprising (or consisting essentially of, or consisting of): (i) an antigen-binding fragment that binds a lineage-specific cell-surface antigen (e.g., CD33); and (ii) a polypeptide that binds a C-type lectin-like natural killer cell receptor.

The present disclosure provides for a fusion polypeptide, comprising (or consisting essentially of, or consisting of): (i) an antigen-binding fragment that binds a lineage-specific cell-surface antigen (e.g., CD33); and (ii) a polypeptide that is capable of stimulating NK cells.

In certain embodiments, the molecule expressed on NK cells is a ligand or receptor expressed on NK cells. The ligand or receptor expressed on NK cells may be a C-type lectin-like NK cell receptor (which may also be called a C-type lectin-like activating receptor, a C-type lectin-like receptor, a killer cell lectin-like receptor, or a C-type lectin-like type II natural killer cell receptor). Non-limiting examples of the molecules expressed on NK cells include NKG2D, CD16, and CD2 (including the members of the CD2-superfamily).

In some embodiments, the polypeptide that binds a molecule expressed on natural killer (NK) cells may comprise an NKG2D ligand or a (functional) NKG2D ligand fragment that binds to NKG2D.

In certain embodiments, the present disclosure provides for a fusion polypeptide, comprising: (i) an antigen-binding fragment that binds CD33 (e.g., human CD33); and (ii) an NKG2D ligand (that binds to NKG2D).

The present disclosure provides a composition comprising: (i) a first antigen-binding moiety (fragment) that binds lineage-specific cell-surface antigen (e.g., CD33); and (ii) a second moiety (e.g., a polypeptide) that binds a molecule expressed on natural killer (NK) cells. For example, the first antigen-binding moiety that binds lineage-specific cell-surface antigen (e.g., CD33), and the second moiety (e.g., a polypeptide) that binds a molecule expressed on natural killer (NK) cells, are present in two separate polypeptides, each of which comprises a dimerization motif (or a polymerization motif) and are brought together (e.g., forming a dimer or polymer) by the dimerization motifs (or polymerization motifs). For example, the first antigen-binding moiety comprises a first dimerization motif, and the second moiety comprises a second dimerization motif, where the first dimerization motif binds to the second dimerization motif.

In certain embodiments, the disclosure provides a composition comprising a first antigen-binding moiety that binds CD33 and a second moiety that binds a molecule expressed on natural killer (NK) cells wherein the first and second moieties each comprise a dimerization motif (or a polymerization motif) and are brought together (e.g., forming a dimer or polymer) by the dimerization motifs (or polymerization motifs).

In certain embodiments, the first antigen-binding moiety that binds lineage-specific cell-surface antigen (e.g., CD33) and the second moiety that binds a molecule expressed on natural killer (NK) cells are polypeptides.

Dimerization motifs include, but are not limited to, IgA, leucine zipper motifs, and an Fc region of an antibody.

The present disclosure provides one or more nucleic acid (or polynucleotide) molecules encoding: (i) a first antigen-binding moiety that binds lineage-specific cell-surface antigen (e.g., CD33); and (ii) a second moiety (e.g., a polypeptide) that binds a molecule expressed on natural killer (NK) cells.

The NKG2D ligand may be a human protein, or can be variants or homologs of the human protein.

Non-limiting examples of the polypeptides that bind a molecule expressed on NK cells (e.g., NKG2D ligands which are ligands that bind to NKG2D) include the ULBP/RAET1 family members (e.g., ULBP1, ULBP2, ULBP3, ULBP4, ULBP5, and ULBP6), MICA, MICB, HCMV UL18, Raet1a, Raet1b, Raet1c, Raet1d, Raet1e, H60b, H60c, homologs thereof, mutants thereof, or fragments thereof.

The present agents, polypeptides, nucleic acid (or polynucleotide) molecules, vectors, cells, compositions and methods allow for targeting a lineage-specific cell-surface antigen (e.g., type 1 or type 2 antigens, such as CD33 or CD19) for treating a hematopoietic malignancy. The methods described herein can maintain surveillance for target cells, including cancer cells that express a lineage-specific cell-surface antigen of interest.

The present compositions and methods may help activate NKG2D-bearing immune effector cells, such as natural killer (NK) cells and/or CD8+ T cells. The present compositions and methods may enhance or prompt a cellular immune response against diseased cells (such as tumor cells) that may induce cytotoxicity (e.g., culminate in the death of the diseased cells such as tumor cells). The present compositions and methods may enhance a subject's immune response, including, but not limited to, one or more of the following: upregulation of natural killer (NK) cell, upregulation of T cell (e.g., gamma delta T cell, alpha beta T cell) function, upregulation of natural killer T (NKT) cell function, and upregulation of B cell function. In some embodiments, upregulation of one or more of NK cell, T cell, natural killer T (NKT) cell, and B cell function includes enhancement and/or endowment of activity capable of inhibiting or decreasing cancer progression.

In some embodiments, inhibiting cancer progression may be accomplished by cytolysis of tumor cells, e.g., by direct induction of tumor cell apoptosis, induction of tumor cell cytolysis through stimulation of intrinsic host antitumor responses, induction of tumor cell apoptosis through stimulation of intrinsic host antitumor responses, inhibition of tumor cell metastasis, inhibition of tumor cell proliferation, and induction of senescence in the tumor cell.

In some embodiments, the antigen-binding fragment binds a lineage-specific cell-surface antigen that is a type 2 lineage-specific cell-surface antigen (e.g., CD33). In some embodiments, the antigen-binding fragment binds a lineage-specific cell-surface antigen that is a type 1 lineage-specific cell-surface antigen (e.g., CD19).

The polypeptide that binds a molecule expressed on natural killer (NK) cells may be a fragment of fragment of ULBP1, ULBP2, ULBP3, ULBP4, ULBP5, ULBP6, MICA, MICB, Raet1a, Raet1b, Raet1c, Raet1d, Raet1e, H60b, H60c, or HCMV UL18, homologs thereof, mutants thereof, or fragments thereof. The polypeptide that binds a molecule expressed on natural killer (NK) cells may be an ectodomain of ULBP1, ULBP2, ULBP3, ULBP4, ULBP5, ULBP6, MICA, MICB, Raet1a, Raet1b, Raet1c, Raet1d, Raet1e, H60b, H60c, HCMV UL18, homologs thereof, mutants thereof, or fragments thereof.

In certain embodiments, the fragment of ULBP1, ULBP2, ULBP3, ULBP4, ULBP5, ULBP6, MICA, MICB, Raet1a, Raet1b, Raet1c, Raet1d, Raet1e, H60b, H60c, or HCMV UL18 comprises an ectodomain of ULBP1, ULBP2, ULBP3, ULBP4, ULBP5, ULBP6, MICA, MICB, Raet1a, Raet1b, Raet1c, Raet1d, Raet1e, H60b, H60c, or HCMV UL18. In certain embodiments, the fragment of ULBP1, ULBP2, ULBP3, ULBP4, ULBP5, ULBP6, MICA, MICB, Raet1a, Raet1b, Raet1c, Raet1d, Raet1e, H60b, H60c, or HCMV UL18 comprises an ectodomain of ULBP1, ULBP2, ULBP3, ULBP4, ULBP5, ULBP6, MICA, MICB, Raet1a, Raet1b, Raet1c, Raet1d, Raet1e, H60b, H60c, HCMV UL18, homologs thereof, mutants thereof, or fragments thereof.

The present disclosure provides one or more nucleic acid (polynucleotide) molecules encoding the present agents or compositions.

Other aspects of the present disclosure provide vectors comprising any of the nucleic acid (or polynucleotide) molecules provided herein. Also within the scope of the present disclosure are polynucleotides encoded by the nucleic acids described herein and cells expressing such polynucleotides.

In some embodiments, the cells can be obtained from a patient having a hematopoietic malignancy. In some embodiments, the cell is a hematopoietic cell, such as a hematopoietic stem cell (e.g., CD34⁺).

Further, the present disclosure provides pharmaceutical compositions comprising the present agents, polypeptides, nucleic acid (or polynucleotide) molecules, vectors, cells, and/or compositions.

Also within the scope of the present disclosure are kits comprising the present agents, polypeptides, nucleic acid (or polynucleotide) molecules, vectors, cells, and/or compositions.

In certain embodiments, the fusion polypeptide comprises: (i) an antigen-binding fragment that binds CD33; and (ii) an ectodomain of ULBP1.

In certain embodiments, antigen-binding fragments include, but are not limited to, Fab, F(ab′)2, Fab′, F(ab)′, Fv, a disulfide linked Fv, single chain Fv (scFv), bivalent scFv (bi-scFv), trivalent scFv (tri-scFv), Fd, dAb fragment, an isolated CDR, diabodies, triabodies, tetrabodies, linear antibodies, single-chain antibody molecules. In some embodiments, scFv comprises a heavy chain variable region (V_{H}), and a light chain variable region (V_{L}).

The fusion polypeptide can be designed to place the functional moieties (an antigen-binding fragment that binds a lineage-specific cell-surface antigen, and a polypeptide that binds a molecule expressed on NK cells) in any order. In certain embodiments, the antigen-binding fragment that binds a lineage-specific cell-surface antigen is located at the N-terminus or C-terminus of the fusion polypeptide. In certain embodiments, the polypeptide that binds a molecule expressed on natural killer (NK) cells is located at the C-terminus or N-terminus of the fusion polypeptide.

In some embodiments, the fusion polypeptide comprises, from N-terminus to C-terminus, a scFv that binds to the lineage-specific cell-surface antigen (e.g., CD33 or CD19), and an ectodomain of ULBP1 (or an ectodomain of ULBP2, ULBP3, ULBP4, ULBP5, ULBP6, MICA, or MICB). In some embodiments, the fusion polypeptide comprises, from N terminus to C terminus, an ectodomain of ULBP1 (or an ectodomain of ULBP2, ULBP3, ULBP4, ULBP5, ULBP6, MICA, or MICB), and a scFv that binds to the lineage-specific cell-surface antigen (e.g., CD33 or CD19).

The fusion polypeptide may further comprise a signal sequence, and/or one or more linkers. In the fusion polypeptide, these functional moieties may be covalently ligated continuously or non-continuously (e.g., they may be separated by linkers (e.g., linker amino acid residues)). The linker may have up to 50, up to 40, up to 30, up to 20, up to 18, up to 15, up to 12, up to 11, or up to 10, amino acid residues in length. In certain embodiments, the linker has about 1, 2, 3, 4, 5, 6, 7, 8, 9, 10-20, 8-10, 8-12, 8-15, 8-20, or 8-30 amino acid residues in length. In certain embodiments, the linker has about 7-10, 7-12, 7-15, 7-20, or 7-30 amino acid residues in length.

One type of derivatized protein is produced by crosslinking two or more polypeptides (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Useful detectable agents with which a protein can be derivatized (or labeled) include fluorescent agents, various enzymes, prosthetic groups, luminescent materials, bioluminescent materials, and radioactive materials. Non-limiting, exemplary fluorescent detectable agents include fluorescein, fluorescein isothiocyanate, rhodamine, and, phycoerythrin. A polypeptide can also be derivatized with detectable enzymes, such as alkaline phosphatase, horseradish peroxidase, beta-galactosidase, acetylcholinesterase, glucose oxidase and the like. A polypeptide can also be derivatized with a prosthetic group (e.g., streptavidin/biotin and avidin/biotin).

The present fusion polypeptide can be derivatized or linked to another functional molecule. For example, present fusion polypeptide can be functionally linked (by chemical coupling, genetic fusion, noncovalent interaction, etc.) to one or more other molecular entities, such as an antibody or antibody fragment, a detectable agent, an immunosuppressant, a cytotoxic agent, a pharmaceutical agent, a protein or peptide that can mediate association with another molecule (such as a streptavidin core region or a polyhistidine tag), amino acid linkers, signal sequences, immunogenic carriers, or ligands useful in protein purification, such as glutathione-S-transferase, histidine tag, and staphylococcal protein A. Cytotoxic agents may include radioactive isotopes, chemotherapeutic agents, and toxins such as enzymatically active toxins of bacterial, fungal, plant, or animal origin, and fragments thereof. The fusion polypeptide may further comprise a fragment (e.g., a tag) useful for polypeptide production and/or detection, including, but not limited to, poly-histidine (e.g., six histidine residues), a maltose binding protein, GST, green fluorescent protein (GFP), hemagglutinin, or alkaline phosphatase, secretion signal peptides (e.g., preprotyrypsin signal sequence), Myc, and/or FLAG.

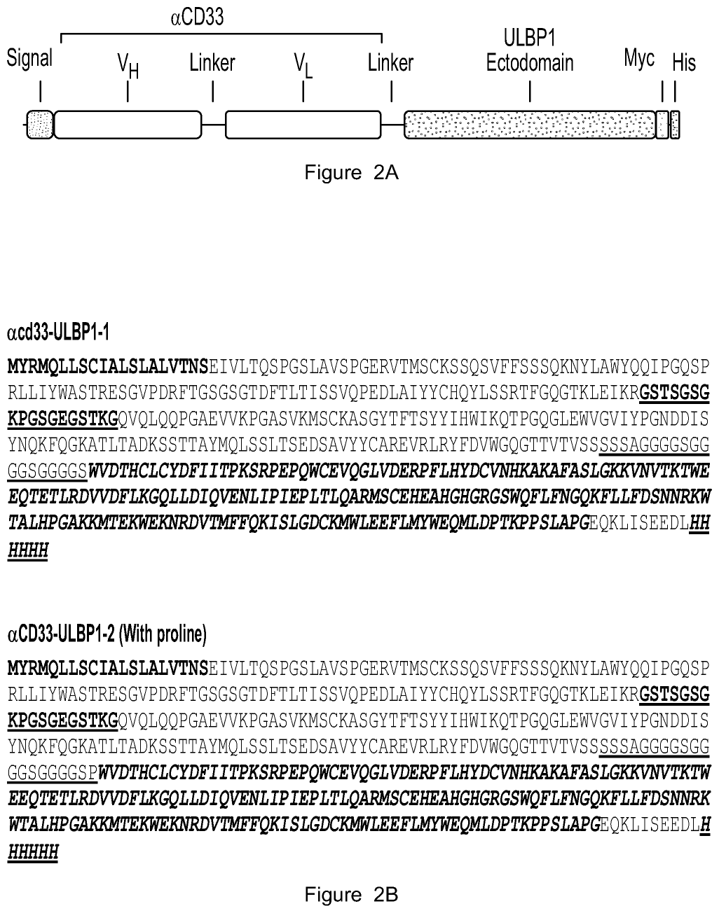

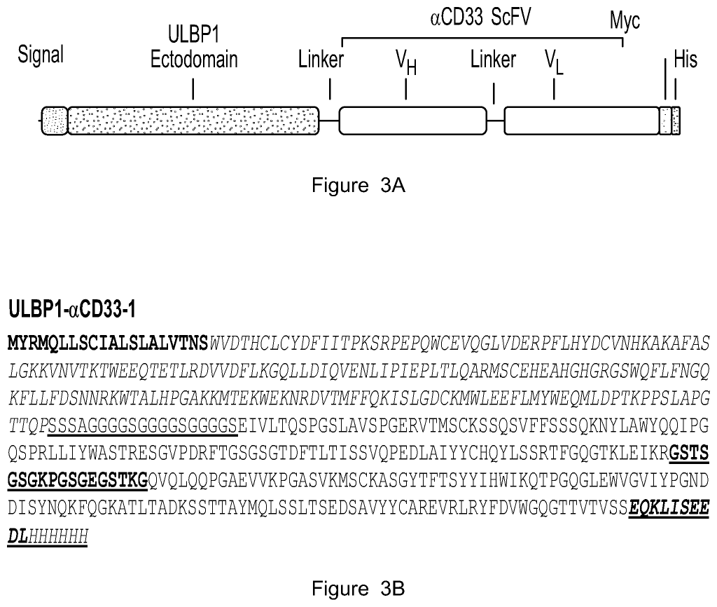

In one embodiment, the fusion polypeptide comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 20, SEQ ID NO: 21, or SEQ ID NO: 22 (FIGS. 2A-2B and 3A-3B).

In one embodiment, the fusion polypeptide comprises a signal sequence comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to an IL2 secretory signal sequence: MYRMQLLSCIALSLALVTNS (SEQ ID NO: 23) (FIGS. 2A-2B and 3A-3B).

In one embodiment, the fusion polypeptide comprises an anti-CD33 scFv comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the anti-CD33 scFv disclosed in U.S. Patent Publication No. 20130078241.

In one embodiment, the fusion polypeptide comprises an antigen-binding fragment that binds CD33, where the antigen-binding fragment comprises a light chain variable region (V_{L}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 10 (FIGS. 2A-2B and 3A-3B):

Anti-CD33 Light Chain variable region (V_{L})
(SEQ ID NO: 10):
EIVLTQSPGSLAVSPGERVTMSCKSSQSVFFSSSQKNYLAWYQQIP
GQSPRLLIYWASTRESGVPDRFTGSGSGTDFTLTISSVQPEDLAIY
YCHQYLSSRTFGQGTKLEIKR

In one embodiment, the fusion polypeptide comprises an antigen-binding fragment that binds CD33, where the antigen-binding fragment comprises a heavy chain variable region (V_{H}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 15 (FIGS. 2A-2B and 3A-3B):

Anti-CD33 Heavy Chain variable region (V_{H})
(SEQ ID NO: 15):
QVQLQQPGAEVVKPGASVKMSCKASGYTFTSYYIHWIKQTPGQGLE
WVGVIYPGNDDISYNQKFQGKATLTADKSSTTAYMQLSSLTSEDSA
VYYCAREVRLRYFDVWGQGTTVTVSS

In certain embodiments, the antigen-binding fragment is a single-chain antibody fragment (scFv) that specifically binds the lineage-specific cell-surface antigen, which can be a human protein, such as human CD33 or CD19. In some embodiments, the scFv binds to human CD33 and comprises a heavy chain variable region, which has the complementary determining regions (CDRs), including CDR1, CDR2 and/or CDR3, comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 16, SEQ ID NO: 17, and/or SEQ ID NO: 18. The scFv may comprise a light chain variable region, which has the CDRs, including CDR1, CDR2 and/or CDR3, comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 11, SEQ ID NO: 12, and/or SEQ ID NO: 13.

In certain embodiments, the antigen-binding fragment that binds the lineage-specific cell-surface antigen (e.g., CD33) comprises a heavy chain variable region, which has the complementary determining regions (CDRs), including CDR1, CDR2 and/or CDR3, comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 16, SEQ ID NO: 17, and/or SEQ ID NO: 18. The antigen-binding fragment that binds the lineage-specific cell-surface antigen (e.g., CD33) may comprise a light chain variable region, which has the CDRs, including CDR1, CDR2 and/or CDR3, comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 11, SEQ ID NO: 12, and/or SEQ ID NO: 13.

In certain embodiments, the antigen-binding fragment that binds a lineage-specific cell-surface antigen comprises a heavy chain variable domain comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 15.

The antigen-binding fragment that binds a lineage-specific cell-surface antigen may comprise a light chain variable domain comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 10.

In certain embodiments, the polypeptide that binds a molecule expressed on natural killer (NK) cells comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence of the full-length, or a fragment, of wildtype ULBP1, ULBP2, ULBP3, ULBP4, ULBP5, ULBP6, MICA, MICB, or HCMV UL18 (including human ULBP1, ULBP2, ULBP3, ULBP4, ULBP5, ULBP6, MICA, MICB, or HCMV UL18), or of the full-length, or a fragment, of the human homolog of Raet1a, Raet1b, Raet1c, Raet1d, Raet1e, H60b, H60c.

In one embodiment, human ULBP1 has a UniProt accession number Q9BZM6 (www.uniprot.org/uniprot/Q9BZM6). In one embodiment, an ectodomain human ULBP1 comprises (or consists essentially of, or consists of) amino acid residues 27 to 216 of Q9BZM6-1.

In one embodiment, the fusion polypeptide comprises a ULBP1 ectodomain comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 1 (FIGS. 2A-2B and 3A-3B):

ULBP1 ectodomain (27 to 216 aa of Q9BZM6-1)
(SEQ ID NO: 1):
WVDTHCLCYDFIITPKSRPEPQWCEVQGLVDERPFLHYDCVNHKAK
AFASLGKKVNVTKTWEEQTETLRDVVDFLKGQLLDIQVENLIPIEP
LTLQARMSCEHEAHGHGRGSWQFLFNGQKFLLFDSNNRKWTALHPG
AKKMTEKWEKNRDVTMFFQKISLGDCKMWLEEFLMYWEQMLDPTKP
PSLAPG

In one embodiment, the fusion polypeptide comprises one or more linkers comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 24, SEQ ID NO: 25, or SEQ ID NO: 26 (FIGS. 2A-2B and 3A-3B): Linker_1 (SEQ ID NO: 24):

### GSTSGSGKPGSGEGSTKG

Linker_2: (GGGGS) × 3 (e.g., separating
anti-CD33 from ULBP1) (SEQ ID NO: 25):
SSSAGGGGSGGGGSGGGGS
Linker_2P: (GGGGS) × 3-(e.g., separating
anti-CD33 from ULBP1) (SEQ ID NO: 26)
SSSAGGGGSGGGGSGGGGSP

In another embodiment, the fusion polypeptide comprises a Myc tag (EQKLISEEDL; SEQ ID NO: 27). In further embodiments, the fusion polypeptide comprises a His6 tag (HHHHHH; SEQ ID NO: 28).

In certain embodiments, the antigen-binding fragment that binds a lineage-specific cell-surface antigen (e.g., CD33) is a derivative, or a modified form, or a variant, of a fragment of the wildtype antigen-binding fragment.

In certain embodiments, the polypeptide that binds a molecule expressed on natural killer (NK) cells is a derivative, or a modified form, or a variant, of a fragment of the wildtype polypeptide.

As used herein, the term variant also denotes any peptide, pseudopeptide (peptide incorporating non-biochemical elements) or protein differing from the wildtype protein or peptide, obtained by one or more genetic and/or chemical modifications. Genetic and/or chemical modification may be understood to mean any mutation, substitution, deletion, addition and/or modification of one or more residues of the protein or peptide considered. Chemical modification may refer to any modification of the peptide or protein generated by chemical reaction or by chemical grafting of biological or non-biological molecule(s) onto any number of residues of the protein.

The present polypeptides or peptides may include variants, analogs, orthologs, homologs and derivatives of amino acids or peptides. The present polypeptides or peptides may contain one or more analogs of amino acids (including, for example, non-naturally occurring amino acids, amino acids which only occur naturally in an unrelated biological system, modified amino acids etc.), peptides with substituted linkages, as well as other modifications known in the art. The present polypeptides or peptides may comprise a peptidomimetic, such as a peptoid. The present polypeptides or peptides may contain one or more amino acid residues modified by, e.g., glycosylation, acylation (e.g., acetylation, formylation, myristoylation, palmitoylation, lipoylation), alkylation (e.g., methylation), isoprenylation or prenylation (e.g., farnesylation, geranylgeranylation), sulfation, amidation, hydroxylation, succinylation, etc. The present polypeptides and agents may be glycosylated, sulfonated and/or phosphorylated and/or grafted to complex sugars or to a lipophilic compound such as, for example, a polycarbon chain or a cholesterol derivative.

Also provided herein are the fusion proteins (chimeric proteins), agents, compositions, nucleic acids encoding such, vectors comprising such nucleic acids, and cells expressing such a chimeric protein or composition.

In another embodiment of this disclosure, polynucleotides encoding one or more of the fusion proteins or agents are provided. For example, polynucleotides encoding any of the proteins described herein are provided, e.g., for recombinant expression and purification. In some embodiments, an isolated polynucleotide comprises one or more sequences encoding the fusion proteins or agents.

In some embodiments, vectors encoding any of the fusion proteins or agents described herein are provided, e.g., for recombinant expression and purification. In some embodiments, the vector comprises or is engineered to include an isolated polynucleotide, e.g., those described herein. Typically, the vector comprises a sequence encoding the fusion protein or agents operably linked to a promoter, such that the fusion protein (or agents) is (are) expressed in a host cell.

In some embodiments, cells are provided, e.g., for recombinant expression and purification of the fusion proteins or agents provided herein. The cells include any cell suitable for recombinant protein expression, for example, cells comprising a genetic construct or vector expressing or capable of expressing an fusion proteins or agents (e.g., cells that have been transformed or transfected with one or more vectors described herein, or cells having genomic modifications, for example, those that express a protein provided herein). Methods for transforming cells, genetically modifying cells, and expressing genes and proteins in such cells are well known in the art, and include those provided by, for example, Green and Sambrook, Molecular Cloning: A Laboratory Manual (4th ed., Cold Spring Harbor Laboratory Press, Cold Spring Harbor, N.Y. (2012)) and Friedman and Rossi, Gene Transfer: Delivery and Expression of DNA and RNA, A Laboratory Manual (1st ed., Cold Spring Harbor Laboratory Press, Cold Spring Harbor, N.Y. (2006)).

Some aspects of this disclosure provide kits comprising the present fusion proteins or agents. In some embodiments, the kit comprises a polynucleotide encoding the present fusion proteins or agents. In some embodiments, the kit comprises a vector for recombinant protein expression, wherein the vector comprises a polynucleotide encoding the present fusion proteins or agents. In some embodiments, the kit comprises a cell that comprises a genetic construct for expressing the present fusion proteins or agents. In some embodiments, the kit comprises an excipient and instructions for using the kit. In some embodiments, the excipient is a pharmaceutically acceptable excipient.

The present disclosure also provides for a method of treating a hematopoietic malignancy. The method may comprise administering to a subject in need thereof an effective amount of the present fusion polypeptide or a polynucleotide encoding the fusion polypeptide. The method may comprise administering to a subject in need thereof an effective amount of the present agents or composition, or a polynucleotide encoding the agents (e.g., a combination of polypeptides) or composition.

Another aspect of the present disclosure provides a method for treating a hematopoietic malignancy (or a hematological neoplasm), the method comprising administering to a subject in need thereof the present agent (e.g., the fusion polypeptide, a nucleic acid molecule or polynucleotide encoding the fusion polypeptide or the present agent) or the present composition).

The present disclosure also relates to methods of using the fusion proteins/polypeptides (protein chimeras, chimeric proteins) to treat hematopoietic malignancies such as myeloid malignancies.

Hematological neoplasms include but not limited to, myeloid malignancies, lymphatic malignancies, malignant histiocytosis and mast cell leukemia, wherein the myeloid malignancies include but not limited to myeloproliferative disorders (MPD), myelodysplastic syndrome (MDS), myelodysplastic/myeloproliferative disorders (MD/MPD) and acute myeloid leukemia (AML); the lymphatic malignancies include but not limited to T/NK cell tumor, B cell tumor and Hodgkin's disease.

The hematopoietic malignancy may be a myeloid malignancy. The subject may have Hodgkin's lymphoma, non-Hodgkin's lymphoma, leukemia, or multiple myeloma. In some examples, the subject has leukemia, which is acute myeloid leukemia, chronic myelogenous leukemia, acute lymphoblastic leukemia, or chronic lymphoblastic leukemia.

Hematopoietic malignancies may refer to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Leukemias include acute myeloid leukemia (AML), acute lymphoid leukemia, chronic myelogenous leukemia, acute lymphoblastic leukemia or chronic lymphoblastic leukemia, and chronic lymphoid leukemia.

Hematological neoplasms include, but are not limited to, myeloid malignancies, lymphatic malignancies, malignant histiocytosis and mast cell leukemia.

In certain embodiments, myeloid malignancies refer to a condition associated with a defect in the proliferation of a hematopoietic cell. In certain embodiments, myeloid malignancies refer to clonal hematological diseases affecting the myeloid blood lineages, including chronic and acute conditions. Myeloid malignancies include myeloproliferative neoplasms, myelodysplastic syndromes and acute myeloid leukemias. A myeloproliferative neoplasm may be primary myelofibrosis (PMF), or essential thrombocythemia (ET). A myelodysplastic syndrome may be refractory anemia with ringed sideroblasts and thrombocythemia (RARS-T). Myeloid malignancies include, but are not limited to, myeloproliferative disorders (MPD), myelodysplastic syndrome (MDS), myelodysplastic/myeloproliferative disorders (MD/MPD), and acute myeloid leukemia (AML).

Lymphatic malignancies include, but are not limited to, T/NK cell tumor, B cell tumor and Hodgkin's disease.

Alternatively or in addition, the methods described herein may be used to treat non-hematopoietic cancers, including without limitation, lung cancer, ear, nose and throat cancer, colon cancer, melanoma, pancreatic cancer, mammary cancer, prostate cancer, breast cancer, ovarian cancer, basal cell carcinoma, biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; liver cancer; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

The present disclosure provides for a vaccine suitable for eliciting an immune response against cancer cells. Method of inhibiting tumor growth by administering the vaccine of the invention to a mammal is also described.

The present composition may be delivered to, or administered to be in contact with, any suitable types of cells. The cell may a eukaryotic cell. The cell may a mammalian cell, such as a human cell or a non-human mammalian cell (e.g., a non-human primate cell). These include a number of cell lines that can be obtained from American Tissue Culture Collection. In certain embodiments, the cell is a tumor cell.

In certain embodiments, the cell is present in a subject (e.g., a mammal). The mammal can be a human or a non-human primate. Non-human primates include, but are not limited to, chimpanzees, cynomolgous monkeys, spider monkeys, and macaques, e.g., Rhesus.

In certain embodiments, the cell may be removed and maintained in tissue culture in a primary, secondary, immortalized or transformed state. In certain embodiments, the cells are cultured cells or cells freshly obtained from a source (e.g., a tissue, an organ, a subject, etc.). The mammalian cell can be primary or secondary which means that it has been maintained in culture for a relatively short time after being obtained from an animal tissue.

### NK Cell Surface Receptor-Binding Peptides

In certain embodiments, the present fusion polypeptide or composition comprises a polypeptide that binds to a molecule expressed on natural killer (NK) cells, such as a C-type lectin-like receptor (e.g., NKG2D).

A C-type lectin-like NK cell receptor may be a receptor expressed on the surface of natural killer cells. Exemplary NK cell receptors of this type include, but are not limited to, NKG2D (GENBANK accession number BC039836), Dectin-1 (GENBANK accession number AJ312373 or AJ312372), Mast cell function-associated antigen (GENBANK accession number AF097358), HNKR-P1A (GENBANK accession number U11276), LLT1 (GENBANK accession number AF133299), CD69 (GENBANK accession number NM_001781), CD69 homolog, CD72 (GENBANK accession number NM_001782), CD94 (GENBANK accession number NM_002262 or NM_007334), KLRF1 (GENBANK accession number NM_016523), Oxidised LDL receptor (GENBANK accession number NM_002543), CLEC-1, CLEC-2 (GENBANK accession number NM_016509), NKG2C (GENBANK accession number AJ001684), NKG2A (GENBANK accession number AF461812), NKG2E (GENBANK accession number AF461157), WUGSC:H_DJ0701016.2, or Myeloid DAP12-associating lectin (MDL-1; GENBANK accession number AJ271684). In particular embodiments, the NK cell receptor is human NKG2D or human NKG2C.

As used herein, the terms “Natural Killer Group 2D”, “NKG2D” and “NKG2D receptor”, also known as KLRK1, refer to an activating cell surface molecule that is found on numerous types of immune cells, particularly NK cells, CD8+ T cells, some CD4+ T cells, and gamma delta T cells. The terms NKG2D and NKG2D receptor include variants, isoforms, and homologs of human NKG2D receptor (see, e.g., the isoforms described in Diefenbach et al., 2002, Nat Immunol. 3(12):1142-9). NKG2D is a type II transmembrane protein with an extracellular C-type (i.e., Ca²⁺-binding) lectin-like domain but lacking the Ca²⁺ binding site. It can form heterodimers with adapter proteins such as DAP10 or DAP12, and recognizes protein ligands that include, but are not limited to, MICA, MICB, ULBP1, ULBP2, ULBP3, ULBP4, ULBP5, and ULBP6.

In certain embodiments, the NKG2D-binding peptide is an agonist of NKG2D. In certain embodiments, the NKG2D-binding peptide is an antagonist of NKG2D. In certain embodiments, the NKG2D-binding peptide is neither an antagonist nor an agonist of NKG2D.

The polypeptide that binds a molecule expressed on NK cells may be a ligand for a NK cell surface receptor, such as a ligand for the NKG2D cell surface receptor. Non-limiting examples of the ligands for NKG2D (or the NKG2D ligands) include, an MHC class I chain-related (MIC) antigen such as MICA and MICB, a UL16 binding protein (ULBP) such as ULBP1, ULBP2, ULBP3, ULBP4, ULBP5, ULBP6, and the like (Bahram (2000) Adv. Immunol. 76:1-60; Cerwenka and Lanier (2001) Immunol. Rev. 181:158-169; Cosman, et al. (2001) Immunity 14:123-133; Kubin, et al. (2001) Eur. J. Immunol. 31:1428-1437). Murine NKG2D ligands include, for example, the retinoic acid early inducible-1 gene products (RAE-1) and minor histocompatibility antigen peptide H60. NK cells can be regulated by interaction of immunomodulating polypeptide ligands with receptors on the NK cell surface. For example, ligands for the NKG2D receptor that can regulate NK cell activity, include chemokines such as muCCL21, and stress-inducible polypeptide ligands such as MHC class I chain-related antigens and ULL16 binding proteins. Murine H60 minor histocompatibility antigen peptide is reported to bind to the NKG2D receptor, as well. See, e.g., Robertson et al. Cell Immunol. 2000; 199(1):8-14; Choi et al. Immunity 2002, 17(5):593-603, and Farag et al., Blood, 2002; 100(6):1935-1947. As used herein, the term “NKG2D ligand” refers to a binding partner that binds specifically to an NKG2D receptor. Exemplary ligands include MICA, MICB, ULBP1, ULBP2, ULBP3, ULBP4, ULBP5, ULBP6, and functional fragments thereof, such as soluble forms of MIC and ULBP proteins.

Table 1 lists exemplary NKG2D binding proteins and domains.

TABLE 1
NKG2D binding proteins and domains

| | | | | NKG2D |
|---|---|---|---|---|
| | | | NKG2D | binding |
| | Gene | Uniport | binding | domain |
| Protein | names | ID | domain* | sequence |
| UL16-binding protein | ULBP1/ | Q9BZM6 | 27-216 | SEQ ID |
| 1 | RAET1I | | | No. 1 |
| UL16-binding protein | ULBP2/ | Q9BZM5 | 26-217 | SEQ ID |
| 2 | RAET1H | | | No. 2 |
| UL16-binding protein | ULBP3/ | Q9BZM4 | 30-217 | SEQ ID |
| 3 | RAET1N | | | No. 3 |
| UL16-binding protein | ULBP4/ | Q8TD07 | 31-225 | SEQ ID |
| 4 | RAET1E | | | No. 4 |
| UL-16 binding protein | ULBP5/ | Q6H3X3 | 26-223 | SEQ ID |
| 5 | RAET1G | | | No. 5 |
| UL16-binding protein | ULBP6/ | Q5VY80 | 26-218 | SEQ ID |
| 6 | RAET1L | | | No. 6 |
| MHC class I | MICA | Q29983 | 24-307 | SEQ ID |
| polypeptide-related | | | | No. 7 |
| sequence A | | | | |
| MHC class I | MICB | Q29980 | 23-309 | SEQ ID |
| polypeptide-related | | | | No. 8 |
| sequence B | | | | |
| Retinoic acid early- | Raet1a | O08602 | 29-229 | SEQ ID |
| inducible protein 1- | | | | NO: 29 |
| alpha | | | | |
| Retinoic acid early- | Raet1b | O08603 | 29-229 | SEQ ID |
| inducible protein 1- | | | | NO: 30 |
| beta | | | | |
| Retinoic acid early- | Raet1c | O08604 | 29-227 | SEQ ID |
| inducible protein 1- | | | | NO: 31 |
| gamma | | | | |
| Retinoic acid early- | Raet1d | Q9JI58 | 29-225 | SEQ ID |
| inducible protein 1- | | | | No. 9 |
| delta | | | | |
| Retinoic acid early- | Raet1e | Q9CZQ6 | 29-225 | SEQ ID |
| inducible protein 1- | | | | NO: 32 |
| epsilon | | | | |
| Histocompatibility | H60b | B1B212 | 25-251 | SEQ ID |
| antigen 60b | | | | NO: 33 |
| Histocompatibility | H60c | B1B213 | 18-177 | SEQ ID |
| antigen 60c | | | | NO: 34 |

*The start and end amino acids numbers are based on the sequence of protein identified by the uniport ID.

The MIC and ULBP proteins act as ligands that bind to C-type lectin-like activating receptor Natural Killer Group 2D (NKG2D) on immune effector cells, including NK cells, NKT cells, alpha beta CD8+ T cells, and gamma delta CD8+ T cells.

As used herein, the term “ULBP protein” refers to members of the MHC class I-related molecules having a characteristic organization for the unprocessed protein that includes a N-terminal signal sequence, centrally located alpha-1 and alpha-2 domains, and a C-terminal cell membrane association domain, which can be a glycosylphosphatidylinositol (GPI) anchoring domain or a transmembrane domain. Some species of ULBP protein have a cytoplasmic domain. Generally, ULBP proteins have weak amino acid sequence identity to MICA/MICB proteins. ULBP family members are ligands for the effector cell receptor NKG2D, and are known to activate NK cells. As used herein. “ULBP protein” includes active variants, isoforms, and species homologs of human ULBP protein, and includes fragments having NKG2D receptor binding activity.

As used herein, the term “ULBP1”, also described as “retinoic acid early transcript 1 protein” or “RAET1”, refers to a member of the MHC class I family, including variants, isoforms, and species homologs of human ULBP1. The protein functions as a ligand for receptor NKG2D. ULBP1 protein activates multiple signaling pathways in primary NK cells. The C terminal membrane association domain in ULBP1 comprises a GPI domain. ULBP1 is weakly homologous with MICA and MICB and has about 55% to 60% amino acid sequence identity to ULBP2 and ULBP3. Exemplary sequence of human ULBP1 is available as NCBI accession no. NP_079494.1. DNA and protein sequences for human ULBP1 have been reported by Cosman et al., Immunity 2001; 14(2):123-133, DNA Accession No. AF304377 in the EMBL database of the European Bioinformatics Institute, Wellcome Trust Genome Campus, Hinxton, Cambridge CB10 1SD, UK. The amino acid sequence of the NKG2D binding domain of human ULBP1 is set forth in SEQ ID NO: 1.

As used herein, the term “ULBP2”, also described as “retinoic acid early transcript 1H protein” or “RAET1H”, refers to a member of the MHC class I family, including variants, isoforms, and species homologs of human ULBP2. The protein functions acts as a ligand for receptor NKG2D. ULBP2 activates multiple signaling pathways in primary NK cells. The C terminal membrane association domain in ULBP2 comprises a GPI domain. ULBP2 is weakly homologous with MICA and MICB and has about 55% and 60% amino acid sequence identity to ULBP1 and ULBP3. Exemplary sequence of human ULBP2 is available as NCBI accession no. NP_079493.1. DNA and protein sequences for human ULBP2 have been reported by Cosman et al., Immunity 2001; 14(2):123-133, DNA Accession No. AF304378 in the EMBL database of the European Bioinformatics Institute, Wellcome Trust Genome Campus, Hinxton, Cambridge CB10 1SD, UK. The amino acid sequence of the NKG2D binding domain of human ULBP2 is set forth in SEQ ID NO: 2.

As used herein, the term “ULBP3”, also described as “retinoic acid early transcript 1N protein” or “RAET1N”, refers to a member of the MHC class I family, including variants, isoforms, and species homologs of human ULBP3. The protein functions as a ligand for receptor NKG2D. The C terminal membrane association domain in ULBP2 comprises a GPI anchoring domain. ULBP3 activates multiple signaling pathways in primary NK cells. ULBP3 is weakly homologous with MICA and MICB. Exemplary sequence of human ULBP3 is available as NCBI accession no. NP_078794.1. DNA and protein sequences for ULBP3 have been reported by Cosman et al., Immunity 2001; 14(2):123-133, DNA Accession No. AF304379 in the EMBL database of the European Bioinformatics Institute, Wellcome Trust Genome Campus, Hinxton, Cambridge CB10 1SD, UK. The amino acid sequence of the NKG2D binding domain of human ULBP3 is set forth in SEQ ID NO: 3.

As used herein, the term “ULBP4”, also described as “retinoic acid early transcript 1E protein” or “RAET1E”, refers to a member of the MHC class I family, including variants, isoforms, and species homologs of human ULBP4. The protein functions as a ligand for receptor NKG2D. The C terminal region of ULBP4 comprises a transmembrane domain and a cytoplasmic domain, (see, e.g., U.S. patent publication US20090274699). ULBP4 is involved in activating NK cells through its binding to receptor NKG2D and induces NK-mediated lysis (see, e.g., Kong et al., 2009, Blood 114(2):310-17). ULBP4 has higher sequence identity to ULBP3 than ULBP1 and ULBP2. Exemplary amino acid sequences of human ULBP4 are available as NCBI accession nos. NP_001230254.1; NP 001230256.1; NP 001230257.1; and NP 631904.1. The amino acid sequence of the NKG2D binding domain of human ULBP4 is set forth in SEQ ID NO: 4.

As used herein, the term “ULBP5”, also described as “retinoic acid early transcript 1G protein” or “RAET1G”, refers to a member of the MHC class I family, including variants, isoforms, and species homologs of human ULBP5. The C-terminal region of the protein has a transmembrane domain and a cytoplasmic domain. ULBP5 is involved in activating NK cells and NK cell-mediated cytotoxicity through its binding to receptor NKG2D. Exemplary sequence of human ULBP5 is available as NCBI accession no. NP_001001788.2. The amino acid sequence of the NKG2D binding domain of human ULBP5 is set forth in SEQ ID NO: 5.

As used herein, the term “ULBP6”, also described as “retinoic acid early transcript 1L protein” or “RAET1L”, refers to a member of the MHC class I family, including variants, isoforms, and species homologs of human ULBP6. ULBP6 contains a GPI anchoring domain, similar to ULBP1, ULBP2, and ULBP3. ULBP6 is involved in activating NK cells and NK cell mediated cytotoxicity through its binding to receptor NKG2D. Exemplary sequence of human ULBP6 is available as NCBI accession no. NP_570970.2. The amino acid sequence of the NKG2D binding domain of human ULBP6 is set forth in SEQ ID NO: 6.

As with MICA and MICB, a known function of ULBP proteins is binding to NKG2D receptor and activating NK cell activity.

MICA is MHC class I chain-related gene A protein (MICA), including variants, isoforms, and homologs of human MICA, and includes fragments of MICA having functional MICA activity. MICA protein comprises three extracellular Ig-like domains, i.e., alpha-1, alpha-2 and alpha-3, a transmembrane domain, and an intracellular domain. The protein is expressed at low levels in cells of the gastric epithelium, endothelial cells and fibroblasts and in the cytoplasm of keratinocytes and monocytes. An exemplary sequence of MICA is available as NCBI Accession Nos. NP_000238.1. Other exemplary MICA sequences can be found in U.S. patent publication 20110311561, incorporated herein by reference.

MICB is MHC class I chain-related gene B protein (MICB), including variants, isoforms, and homologs of human MICB, and includes fragments of MICB having functional MICB activity. MICB has about 84% sequence identity to MICA. MICB protein comprises three extracellular Ig-like domains, i.e., alpha-1, alpha-2 and alpha-3, a transmembrane domain, and an intracellular domain. An exemplary sequence of MICB is available as UniProtKB accession number Q29980.1. Other exemplary MICB sequences can be found in U.S. patent publication 20110311561, incorporated herein by reference.

In certain embodiments, the polypeptide that binds a molecule expressed on natural killer (NK) cells comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 29, SEQ ID NO: 30, SEQ ID NO: 31, SEQ ID NO: 32, SEQ ID NO: 33, or SEQ ID NO: 34.

The NKG2D ligands (ligands for the NKG2D receptor) may also include an anti-NKG2D antibody or its fragment (e.g., an antigen-binding portion or fragment thereof), including, but not limited to, all or part of antibody that specifically recognizes or binds to NKG2D. Such antibodies can be monoclonal or polyclonal antibodies. Antibodies can also be variant antibodies, such as chimeric antibodies, humanized antibodies, single chain antibodies, and hybrid antibodies comprising immunoglobulin chains capable of binding NKG2D. In particular embodiments, the antibody comprises a single chain variable fragment. In particular embodiments, the antibody is 16F16, 16F31, MS, or 21F2, as set forth in U.S. Pat. No. 7,879,985, which is hereby incorporated by reference. The antibody fragment can be any suitable fragment as discussed herein.

In certain embodiments, the polypeptide that binds a molecule expressed on natural killer (NK) cells has up to or about 500, up to or about 490, up to or about 480, up to or about 470, up to or about 460, up to or about 450, up to or about 440, up to or about 430, up to or about 420, up to or about 410, up to or about 400, up to or about 390, up to or about 380, up to or about 370, up to or about 360, up to or about 350, up to or about 340, up to or about 330, up to or about 320, up to or about 310, up to or about 200, up to or about 190, up to or about 180, up to or about 170, up to or about 160, up to or about 150, up to or about 140, up to or about 130, up to or about 120, up to or about 110, up to or about 100, up to or about 90, up to or about 80, up to or about 70, up to or about 60, up to or about 50, up to or about 40, up to or about 30, up to or about 20, up to or about 15, or up to or about 10, amino acid residues in length. In certain embodiments, the polypeptide that binds a molecule expressed on natural killer (NK) cells has about 100-200, 80-210, 80-250, 150-250, 100-30, 50-200, 150-250, 150-300, or 150-190 amino acid residues in length.

### Lineage-Specific Cell-Surface Antigens

Aspects of the disclosure provide agents targeting a lineage-specific cell-surface antigen, for example on a target cancer cell. Such an agent may comprise an antigen-binding fragment that binds and targets the lineage-specific cell-surface antigen. In some instances, the antigen-binding fragment can be a single chain antibody (scFv) specifically binding to the lineage-specific antigen.

As used herein, the terms “lineage-specific cell-surface antigen” and “cell-surface lineage-specific antigen” may be used interchangeably and refer to any antigen that is sufficiently present on the surface of a cell and is associated with one or more populations of cell lineage(s). For example, the antigen may be present on one or more populations of cell lineage(s) and absent (or at reduced levels) on the cell-surface of other cell populations.

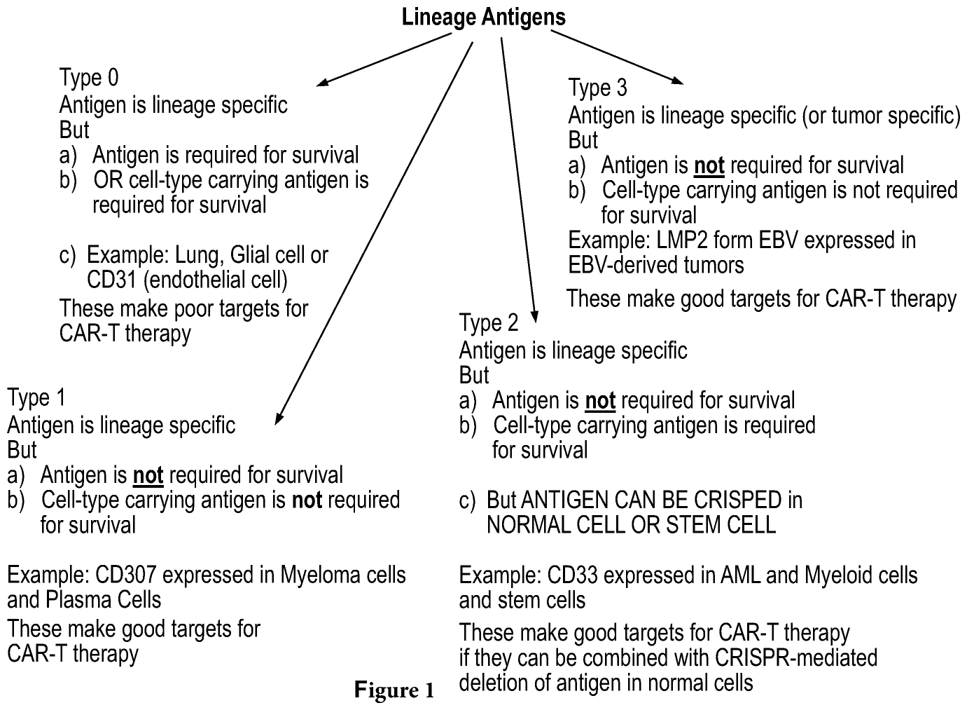

In general, lineage-specific cell-surface antigens can be classified based on a number of factors such as whether the antigen and/or the populations of cells that present the antigen are required for survival and/or development of the host organism. A summary of exemplary types of lineage-specific antigens is provide in Table 2 below. See also FIG. 1.

TABLE 2
Classification of Lineage Specific Antigens

| Type of | |
|---|---|
| Lineage | |
| Specific | |
| Antigen | Characteristics of the Lineage Specific Antigen |
| Type 0 | a) antigen is required for survival of an organism and |
| | b) cell type carrying type 0 antigen is required for survival of an |
| | organism and is not unique to a tumor, or tumor-associated virus |
| Type 1 | a) antigen is not required for survival of an organism and |
| | b) cell type carrying type 1 antigen is not required for survival of |
| | an organism |
| Type 2 | a) antigen is not required for survival of an organism and |
| | b) cell type carrying type 2 antigen is required for the survival of |
| | an organism |
| Type 3 | a) antigen is not required for the survival of an organism and |
| | b) cell type carrying antigen is not required for survival of an |
| | organism |
| | c) The antigen is unique to a tumor, or a tumor associated virus. |
| | An example is the LMP-2 antigen in EBV infected cells, |
| | including EBV infected tumor cells (Nasopharyngeal carcinoma |
| | and Burkitts Lymphoma) |

Lineage specific antigens of type 1 class may be expressed in a wide variety of different tissues, including, ovaries, testes, prostate, breast, endometrium, and pancreas. In some embodiments, the agent targets a cell-surface lineage-specific antigen that is a type 1 antigen.

In some embodiments, the agent targets a cell-surface lineage-specific antigen that is a type 2 antigen. For example, CD33 is a type 2 antigen expressed in both normal myeloid cells as well as in Acute Myeloid Leukemia (AML) cells (Dohner et al., NEJM 373:1136 (2015)).

A wide variety of antigens may be targeted by the methods and compositions of the present disclosure. Monoclonal antibodies to these antigens may be purchased commercially or generated using standard techniques, including immunization of an animal with the antigen of interest followed by conventional monoclonal antibody methodologies e.g., the standard somatic cell hybridization technique of Kohler and Milstein, Nature (1975) 256: 495, as discussed above. The antibodies or nucleic acids encoding for the antibodies may be sequenced using any standard DNA or protein sequencing techniques.

In some embodiments, the cell-surface lineage-specific antigen that is targeted using the methods and compositions described herein is a cell-surface lineage-specific antigen of leukocytes or a subpopulation of leukocytes. In some embodiments, the cell-surface lineage-specific antigen is an antigen that is associated with myeloid cells. In some embodiments, the cell-surface lineage-specific antigen is a cluster of differentiation antigens (CDs). Examples of CD antigens include, without limitation, CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, LD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85L CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD144, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, 306, CD307a, CD307b, CD307c, D307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 and CD363. See www.bdbiosciences.com/documents/BD_Reagents_CDMarkerHuman_Poster.pdf.

In some embodiments, the cell-surface lineage-specific antigen is CD19, CD20, CD11, CD123, CD56, CD34, CD14, CD33, CD66b, CD41, CD61, CD62, CD235a, CD146, CD326, LMP2, CD22, CD52, CD10, CD3/TCR, CD79/BCR, and CD26. In some embodiments, the cell-surface lineage-specific antigen is CD33 or CD19.

Alternatively or in addition, the cell-surface lineage-specific antigen may be a cancer antigen, for example a cell-surface lineage-specific antigen that is differentially present on cancer cells. In some embodiments, the cancer antigen is an antigen that is specific to a tissue or cell lineage. Examples of cell-surface lineage-specific antigen that are associated with a specific type of cancer include, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen. In some embodiments, the cell-surface antigen CD33 and is associated with AML cells.

In certain embodiments, the antigen-binding fragment that binds a lineage-specific cell-surface antigen (e.g., CD33) has up to or about 500, up to or about 490, up to or about 480, up to or about 470, up to or about 460, up to or about 450, up to or about 440, up to or about 430, up to or about 420, up to or about 410, up to or about 400, up to or about 390, up to or about 380, up to or about 370, up to or about 360, up to or about 350, up to or about 340, up to or about 330, up to or about 320, up to or about 310, up to or about 200, up to or about 190, up to or about 180, up to or about 170, up to or about 160, up to or about 150, up to or about 140, up to or about 130, up to or about 120, up to or about 110, up to or about 100, up to or about 90, up to or about 80, up to or about 70, up to or about 60, up to or about 50, up to or about 40, up to or about 30, up to or about 20, up to or about 15, or up to or about 10, amino acid residues in length. In certain embodiments, the antigen-binding fragment that binds a lineage-specific cell-surface antigen (e.g., CD33) has about 100-200, 80-210, 80-250, 150-250, 100-30, 50-200, 150-250, 150-300, 300-400, 200-400, 400-500, or 150-190 amino acid residues in length.

In certain embodiments, the antigen-binding fragment that binds a lineage-specific cell-surface antigen (e.g., CD33) comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 10 and/or SEQ ID NO: 15.

In some embodiments, the agents targeting a cell-surface lineage-specific antigen is an antibody-drug conjugate (ADC). As will be evident to one of ordinary skill in the art, the term “antibody-drug conjugate” can be used interchangeably with “immunotoxin” and refers to a fusion molecule comprising an antibody (or antigen-binding fragment thereof) conjugated to a toxin or drug molecule. Binding of the antibody to the corresponding antigen allows for delivery of the toxin or drug molecule to a cell that presents the antigen on the its cell surface (e.g., target cell), thereby resulting in death of the target cell.

In some embodiments, the agent is an antibody-drug conjugate. In some embodiments, the antibody-drug conjugate comprises an antigen-binding fragment and a toxin or drug that induces cytotoxicity in a target cell. In some embodiments, the antibody-drug conjugate targets a type 2 antigen. In some embodiments, the antibody-drug conjugate targets CD33 or CD19.

Toxins or drugs compatible for use in antibody-drug conjugate are well known in the art and will be evident to one of ordinary skill in the art. See, e.g., Peters et al. Biosci. Rep. (2015) 35(4): e00225. In some embodiments, the antibody-drug conjugate may further comprise a linker (e.g., a peptide linker, such as a cleavable linker) attaching the antibody and drug molecule.

An ADC described herein may be used as a follow-on treatment to subjects who have been undergone the combined therapy as described herein.

### Antigen-Binding Fragment

The antigen-binding fragment may be an antibody fragment. The antibody or antibody fragment may be any of the immunoglobulin classes (e.g., IgA, IgD, IgE, IgG, and IgM) and subclasses, so long as they are capable of binding NKG2D. In certain embodiments, the antibody fragment has an antigen-binding portion. In certain embodiments, antibody fragments include, but are not limited to, Fab, F(ab′)2, Fab′, F(ab)′, Fv, a disulfide linked Fv, single chain Fv (scFv), bivalent scFv (bi-scFv), trivalent scFv (tri-scFv), Fd, dAb fragment (e.g., Ward et al., Nature, 341:544-546 (1989)), an isolated CDR, diabodies, affibodies, triabodies, tetrabodies, linear antibodies, single-chain antibody molecules. Single chain antibodies produced by joining antibody fragments using recombinant methods, or a synthetic linker, are also encompassed by the present disclosure. Bird et al. Science, 1988, 242:423-426. Huston et al., Proc. Natl. Acad. Sci. USA, 1988, 85:5879-5883. Antibody fragments comprise only a portion of an intact antibody, generally including an antigen binding site of the intact antibody and thus retaining the ability to bind antigen. Examples of antibody fragments encompassed by the present invention include: the Fab fragment, having a light chain variable domain (V_{L}), light chain constant domain (C_{L}), heavy chain variable domain (V_{H}), and heavy chain constant domain (C_{H}); the Fab′ fragment, which is a Fab fragment having one or more cysteine residues at the C-terminus of the C_{H} domain; the Fd fragment having V_{H} and C_{H} domains; the Fd′ fragment having V_{H} and C_{H} domains and one or more cysteine residues at the C-terminus of the C_{H} domain; the Fv fragment having the V_{L} and V_{H} domains of a single arm of an antibody; the dAb fragment (Ward et al., “Binding Activities of a Repertoire of Single Immunoglobulin Variable Domains Secreted from Escherichia coli,” Nature 341:544-546 (1989), which is hereby incorporated by reference in its entirety) which consists of a V_{H} domain; isolated CDR regions; F(ab′)2 fragments, a bivalent fragment including two Fab′ fragments linked by a disulphide bridge at the hinge region; single chain antibody molecules (Bird et al., “Single-Chain Antigen Binding Proteins,” Science 242:423-426 (1988); and Huston et al., “Protein Engineering of Antibody Binding Sites: Recovery of Specific Activity in an Anti-Digoxin Single-Chain Fv Analogue Produced in Escherichia coli,” PNAS 85:5879-5883 (1988), which are hereby incorporated by reference in their entirety); diabodies with two antigen binding sites, comprising a V_{H} domain connected to a V_{L} domain in the same polypeptide chain (see, e.g., WO 93/11161 to Whitlow et al. and Hollinger et al., “Diabodies: Small Bivalent and Bispecific Antibody Fragments,” PNAS 90:6444-6448 (1993), which are hereby incorporated by reference in their entirety); affibodies which are triple helix high affinity peptides (see, e.g., Nygren P., Alternative binding proteins: Affibody binding proteins developed from a small three-helix bundle scaffold, FEBS Journal 275 (2008) 2668-2676, which is hereby incorporated by reference in its entirety), and linear antibodies comprising a pair of tandem Fd segments (VH-CH1-VH-CH1) which, together with complementary light chain polypeptides, form a pair of antigen binding regions (Zapata et al., “Engineering Linear F(ab′)2 Fragments for Efficient Production in Escherichia coli and Enhanced Antiproliferative Activity,” Protein Eng. 8(10): 1057-1062 (1995); U.S. Pat. Nos. 5,641,870; 8,580,755, which are hereby incorporated by reference in their entirety).

Any antibody or an antigen-binding fragment thereof can be used for constructing the agent that targets a lineage-specific cell-surface antigen as described herein. Such an antibody or antigen-binding fragment can be prepared by a conventional method, for example, the hybridoma technology or recombinant technology.

For example, antibodies specific to a lineage-specific antigen of interest can be made by the conventional hybridoma technology. The lineage-specific antigen, which may be coupled to a carrier protein such as KLH, can be used to immunize a host animal for generating antibodies binding to that complex. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the TCR-like monoclonal antibodies described herein. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of binding to a lineage-specific antigen. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the lineage-specific antigen. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a lineage-specific antigen can be identified from the library following routine procedures. Positive clones can be subjected to further screening to identify those that bind lineage-specific antigen.

In some instances, lineage-specific antigen of interest is CD33 and the antigen-binding fragment specifically binds CD33, for example, human CD33. Amino acid and nucleic acid sequences of an exemplary heavy chain variable region and light chain variable region of an anti-human CD33 antibody are provided below. The CDR sequences are shown in boldface and underlined in the amino acid sequences.

Amino acid sequence of anti-CD33 Heavy Chain
Variable Region
(SEQ ID NO: 15)
QVQLQQPGAEVVKPGASVKMSCKASGYTFTSYYIHWIKQTPGQGLE
WVGVIYPGNDDISYNQKFQGKATLTADKSSTTAYMQLSSLTSEDSA
VYYCAREVRLRYFDVWGQGTTVTVSS
Nucleic acid sequence of anti-CD33 Heavy Chain
Variable Region
(SEQ ID NO: 19)
CAGGTGCAGCTGCAGCAGCCCGGCGCCGAGGTGGTGAAGCCCGGCG
CCAGCGTGAAGATGAGCTGCAAGGCCAGCGGCTACACCTTCACCAG
CTACTACATCCACTGGATCAAGCAGACCCCCGGCCAGGGCCTGGAG
TGGGTGGGCGTGATCTACCCCGGCAACGACGACATCAGCTACAACC
AGAAGTTCCAGGGCAAGGCCACCCTGACCGCCGACAAGAGCAGCAC
CACCGCCTACATGCAGCTGAGCAGCCTGACCAGCGAGGACAGCGCC
GTGTACTACTGCGCCAGGGAGGTGAGGCTGAGGTACTTCGACGTGT
GGGGCCAGGGCACCACCGTGACCGTGAGCAGC
Amino acid sequence of anti-CD33 Light Chain
Variable Region
(SEQ ID NO: 10)
EIVLTQSPGSLAVSPGERVTMSCKSSQSVFFSSSQKNYLAWYQQIP
GQSPRLLIYWASTRESGVPDRFTGSGSGTDFTLTISSVQPEDLAIY
YCHQYLSSRTFGQGTKLEIKR
Nucleic acid sequence of anti-CD33 Heavy Chain
Variable Region
(SEQ ID NO: 14)
GAGATCGTGCTGACCCAGAGCCCCGGCAGCCTGGCCGTGAGCCCCG
GCGAGAGGGTGACCATGAGCTGCAAGAGCAGCCAGAGCGTGTTCTT
CAGCAGCAGCCAGAAGAACTACCTGGCCTGGTACCAGCAGATCCCC
GGCCAGAGCCCCAGGCTGCTGATCTACTGGGCCAGCACCAGGGAGA
GCGGCGTGCCCGACAGGTTCACCGGCAGCGGCAGCGGCACCGACTT
CACCCTGACCATCAGCAGCGTGCAGCCCGAGGACCTGGCCATCTAC
TACTGCCACCAGTACCTGAGCAGCAGGACCTTCGGCCAGGGCACCA
AGCTGGAGATCAAGAGG

The anti-CD33 antibody binding fragment for use in constructing the agent that targets CD33 as described herein may comprise the same heavy chain and/or light chain CDR regions as those in SEQ ID NOs:16-18 and SEQ ID NOs: 11-13. Such antibodies may comprise amino acid residue variations in one or more of the framework regions. In some instances, the anti-CD33 antibody fragment may comprise a heavy chain variable region that shares at least 70% sequence identity (e.g., 75%, 80%, 85%, 90%, 95%, or higher) with SEQ ID NO:15 and/or may comprise a light chain variable region that shares at least 70% sequence identity (e.g., 75%, 80%, 85%, 90%, 95%, or higher) with SEQ ID NO:10.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the present disclosure. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

### Vectors

The present disclosure provides for a nucleic acid/polynucleotide encoding the fusion polypeptide or agents. The nucleic acid may be deoxyribonucleic acid (DNA), ribonucleic acid (RNA) or a DNA/RNA hybrid. The nucleic acid may be linear or circular (such as a plasmid). The nucleic acid may be single-stranded, double-stranded, branched or modified by the ligation of non-nucleic acid molecules. The nucleic acids include nucleic acids produced by recombinant technology.

In certain embodiments, the nucleic acid is a plasmid DNA including a coding sequence for the fusion polypeptide or agents, together with flanking regulatory sequences effective to cause the expression of the fusion polypeptide or agents in cells. Examples of flanking regulatory sequences are a promoter sequence sufficient to initiate transcription and a terminator sequence sufficient to terminate the gene product, by termination of transcription or translation. Suitable transcriptional or translational enhancers can be included in the vector to further assist the expression of the fusion polypeptide or agents.

The nucleic acid may be contained within an expression vector. Thus, for example, a nucleic acid sequence may be included in any one of a variety of expression vectors for expressing one or more polypeptides, and more than one nucleic acid may be included in one expression vector. Alternatively, parts of one gene or nucleic acid may be included in separate vectors. In some embodiments, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and derivatives of viral DNA).

Vectors of the present disclosure can drive the expression of one or more sequences in mammalian cells using a mammalian expression vector. Examples of mammalian expression vectors include pCDM8 (Seed, Nature (1987) 329: 840) and pMT2PC (Kaufman, et al., EMBO J. (1987) 6: 187). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989.

The vectors of the present disclosure may direct expression of the nucleic acid preferentially in a particular cell type (e.g., tissue-specific regulatory elements are used to express the nucleic acid). Such regulatory elements include promoters that may be tissue-specific or cell type-specific. The term “tissue-specific” as it applies to a promoter refers to a promoter that is capable of directing selective expression of a nucleotide sequence of interest to a specific type of tissue in the relative absence of expression of the same nucleotide sequence of interest in a different type of tissue. The term “cell type-specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type-specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Conventional viral and non-viral based gene transfer methods can be used to introduce nucleic acids in mammalian cells or target tissues. Such methods can be used to administer nucleic acids encoding the present agents/polypeptides to cells in culture, or in a subject. Non-viral vector delivery systems include DNA plasmids, RNA (e.g., a transcript of a vector described herein), naked nucleic acid, and nucleic acid complexed with a delivery vehicle. Viral vector delivery systems include DNA and RNA viruses, which have either episomal or integrated genomes after delivery to the cell.

Viral vectors can be administered directly to patients (in vivo) or they can be used to manipulate cells in vitro or ex vivo, where the modified cells may be administered to patients. In one embodiment, the present disclosure utilizes viral based systems including, but not limited to retroviral, lentivirus, adenoviral, adeno-associated and herpes simplex virus vectors for gene transfer. Furthermore, the present disclosure provides vectors capable of integration in the host genome, such as retrovirus or lentivirus.

The vectors of the present disclosure may be delivered to the eukaryotic cell in a subject. Any of the chimeric proteins described herein can be prepared by routine methods, such as recombinant technology. Methods for preparing the chimeric proteins herein involve generation of a nucleic acid that encodes a polypeptide comprising each of the fragments/domains/moieties of the chimeric proteins, including the antigen-binding fragment and the polypeptide that binds a molecule expressed on natural killer (NK) cells. In some embodiments, a nucleic acid encoding each of the components of chimeric protein are joined together using recombinant technology.

Sequences of each of the components of the chimeric proteins may be obtained via routine technology, e.g., PCR amplification from any one of a variety of sources known in the art. In some embodiments, sequences of one or more of the components of the chimeric proteins are obtained from a human cell. Alternatively, the sequences of one or more components of the chimeric proteins can be synthesized. Sequences of each of the components (e.g., fragments/domains/moieties) can be joined directly or indirectly (e.g., using a nucleic acid sequence encoding a peptide linker) to form a nucleic acid sequence encoding the chimeric protein, using methods such as PCR amplification or ligation. Alternatively, the nucleic acid encoding the chimeric protein may be synthesized. In some embodiments, the nucleic acid is DNA. In other embodiments, the nucleic acid is RNA.

Mutation of one or more residues within one or more of the components of the chimeric protein (e.g., the antigen-binding fragment, etc.), prior to or after joining the sequences of each of the components. In some embodiments, one or more mutations in a component of the chimeric protein may be made to modulate (increase or decrease) the affinity of the component for a target (e.g., the antigen-binding fragment for the target antigen) and/or modulate the activity of the component.

Any of the chimeric proteins described herein can be introduced into a suitable cell for expression via conventional technology.

To express the chimeric proteins, expression vectors for stable or transient expression of the chimeric proteins may be constructed via conventional methods as described herein. For example, nucleic acids encoding the chimeric proteins may be cloned into a suitable expression vector, such as a viral vector in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the chimeric proteins. The synthetic linkers may contain nucleic acid sequences that correspond to a particular restriction site in the vector. The selection of expression vectors/plasmids/viral vectors would depend on the type of host cells for expression of the chimeric proteins, but should be suitable for integration and replication in eukaryotic cells.

A variety of promoters can be used for expression of the chimeric proteins described herein, including, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters for expression of the chimeric proteins include any constitutively active promoter. Alternatively, any regulatable promoter may be used, such that its expression can be modulated.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric protein. See section VI below. Suitable vectors and methods for producing vectors containing transgenes are well known and available in the art. Examples of the preparation of vectors for expression of chimeric proteins can be found, for example, in US2014/0106449, herein incorporated by reference in its entirety.

In some embodiments, the chimeric protein or the nucleic acid encoding said chimeric protein is a DNA molecule. In some embodiments, chimeric protein or the nucleic acid encoding said chimeric protein is a DNA vector. In some embodiments, the nucleic acid encoding the chimeric protein is an RNA molecule.

Any of the vectors comprising a nucleic acid sequence that encodes a chimeric protein described herein is also within the scope of the present disclosure. Such a vector may be delivered into host cells by a suitable method. Methods of delivering vectors to cells are well known in the art and may include DNA, RNA, or transposon electroporation, transfection reagents such as liposomes or nanoparticles to delivery DNA, RNA, or transposons; delivery of DNA, RNA, or transposons or protein by mechanical deformation (see, e.g., Sharei et al. Proc. Natl. Acad. Sci. USA (2013) 110(6): 2082-2087); or viral transduction. In some embodiments, the vectors for expression of the chimeric proteins are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984 and WO 95/00655). In some embodiments, the vectors for expression of the chimeric proteins are retroviruses. In some embodiments, the vectors for expression of the chimeric proteins are lentiviruses. In some embodiments, the vectors for expression of the chimeric proteins are adeno-associated viruses.

In examples in which the vectors encoding chimeric proteins are introduced to the host cells using a viral vector, viral particles that are capable of infecting the cells and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to contacting the viral particles with the cells.

### Therapeutic Methods

The present nucleic acids/polynucleotides/vectors encoding the fusion polypeptides or agents may be administered to a subject to treat a condition such as hematopoietic malignancy. The present fusion polypeptides, agents or compositions may be administered to a subject. As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human patient having a hematopoietic malignancy.

In some embodiments, the present vectors, fusion polypeptides or agents may be mixed with a pharmaceutically acceptable carrier to form a pharmaceutical composition, which is also within the scope of the present disclosure.

To perform the methods described herein, an effective amount of the present composition may be administered to a subject in need of the treatment. As used herein the term “effective amount” may be used interchangeably with the term “therapeutically effective amount” and refers to that quantity of a vector, a fusion polypeptide, an agent, or pharmaceutical composition that is sufficient to result in a desired activity upon administration to a subject in need thereof. Within the context of the present disclosure, the term “effective amount” refers to that quantity of a vector, a fusion polypeptide, an agent, or pharmaceutical composition that is sufficient to delay the manifestation, arrest the progression, relieve or alleviate at least one symptom of a disorder treated by the methods of the present disclosure.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject is a human patient having a hematopoietic malignancy.

In some embodiments, the present composition is administered to a subject in an amount effective in to reduce the number of target cells (e.g., cancer cells) by least 20%, e.g., 50%, 80%, 100%, 2-fold, 5-fold, 10-fold, 20-fold, 50-fold, 100-fold or more.

In one embodiment, the present composition is administered to a subject (e.g., human patient) as an initial dose. One or more subsequent administrations of the present composition may be provided to the patient at intervals of 15 days, 14, 13, 12, 11, 10, 9, 8, 7, 6, 5, 4, 3, or 2 days after the previous administration. More than one dose of the present composition can be administered to the subject per week, e.g., 2, 3, 4, or more administrations of the agent. The subject may receive more than one doses of the present composition per week, followed by a week of no administration of the agent, and finally followed by one or more additional doses of the present composition (e.g., more than one administration of the present composition per week). The present composition may be administered every other day for 3 administrations per week for two, three, four, five, six, seven, eight or more weeks.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

In some embodiments, the present fusion polypeptide/agent recognizes (binds) a target cell expressing the cell-surface lineage-specific antigen for targeting killing.

The efficacy of the present therapeutic methods may be assessed by any method known in the art and would be evident to a skilled medical professional. For example, the efficacy of the therapy may be assessed by survival of the subject or cancer burden in the subject or tissue or sample thereof. In some embodiments, the efficacy of the therapy is assessed by quantifying the number of cells belonging to a particular population or lineage of cells. In some embodiments, the efficacy of the therapy is assessed by quantifying the number of cells presenting the cell-surface lineage-specific antigen.

The present composition may be administered to a subject in combination with a second therapy. The present composition may be administered prior to administration of the second therapy. In some embodiments, the present composition is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the the second therapy.

In some embodiments, the second therapy is administered prior to the administration of the present composition. In some embodiments, the second therapy is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the present composition.

In some embodiments, the present composition and the second therapy are administered at substantially the same time. In some embodiments, the present composition is administered, and the patient is assessed for a period of time, after which the second therapy is administered. In some embodiments, the second therapy is administered, and the patient is assessed for a period of time, after which the present composition is administered.

Also within the scope of the present disclosure are multiple administrations (e.g., doses) of the present composition. In some embodiments, the present composition is administered to the subject once. In some embodiments, the present composition is administered to the subject more than once (e.g., at least 2, 3, 4, 5, or more times). In some embodiments, the present composition is administered to the subject at a regular interval, e.g., every six months.

In some embodiments, the subject is a human subject having a hematopoietic malignancy. As used herein a hematopoietic malignancy refers to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Leukemias include acute myeloid leukaemia, acute lymphoid leukemia, chronic myelogenous leukaemia, acute lymphoblastic leukemia or chronic lymphoblastic leukemia, and chronic lymphoid leukemia.

In some embodiments, the leukemia is acute myeloid leukaemia (AML). AML is characterized as a heterogeneous, clonal, neoplastic disease that originates from transformed cells that have progressively acquired critical genetic changes that disrupt key differentiation and growth-regulatory pathways. (Dohner et al., NEJM, (2015) 373:1136). CD33 glycoprotein is expressed on the majority of myeloid leukemia cells as well as on normal myeloid and monocytic precursors and has been considered to be an attractive target for AML therapy (Laszlo et al., Blood Rev. (2014) 28(4):143-53). While clinical trials using anti CD33 monoclonal antibody based therapy have shown improved survival in a subset of AML patients when combined with standard chemotherapy, these effects were also accompanied by safety and efficacy concerns.

Alternatively or in addition, the methods described herein may be used to treat non-hematopoietic cancers, including without limitation, lung cancer, ear, nose and throat cancer, colon cancer, melanoma, pancreatic cancer, mammary cancer, prostate cancer, breast cancer, ovarian cancer, basal cell carcinoma, biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; liver cancer; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

Carcinomas are cancers of epithelial origin. Carcinomas intended for treatment with the methods of the present disclosure include, but are not limited to, acinar carcinoma, acinous carcinoma, alveolar adenocarcinoma (also called adenocystic carcinoma, adenomyoepithelioina, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Huirthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypemephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum. In preferred embodiments, the methods of the present disclosure are used to treat subjects having cancer of the breast, cervix, ovary, prostate, lung, colon and rectum, pancreas, stomach or kidney.

Sarcomas are mesenchymal neoplasms that arise in bone and soft tissues. Different types of sarcomas are recognized and these include: liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., non-bone) Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

In some embodiments, the cancer to be treated can be a refractory cancer. A “refractory cancer,” as used herein, is a cancer that is resistant to the standard of care prescribed. These cancers may appear initially responsive to a treatment (and then recur), or they may be completely non-responsive to the treatment. The ordinary standard of care will vary depending upon the cancer type, and the degree of progression in the subject. It may be a chemotherapy, or surgery, or radiation, or a combination thereof. Those of ordinary skill in the art are aware of such standards of care. Subjects being treated according to the present disclosure for a refractory cancer therefore may have already been exposed to another treatment for their cancer. Alternatively, if the cancer is likely to be refractory (e.g., given an analysis of the cancer cells or history of the subject), then the subject may not have already been exposed to another treatment. Examples of refractory cancers include, but are not limited to, leukemia, melanomas, renal cell carcinomas, colon cancer, liver (hepatic) cancers, pancreatic cancer, Non-Hodgkin's lymphoma and lung cancer.

Any of the present vectors, fusion polypeptides or agents described herein may be administered in a pharmaceutically acceptable carrier or excipient as a pharmaceutical composition.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

### Kits

Also within the scope of the present disclosure are kits for use of the present fusion polypeptides, agents, vectors, and/or compositions. Such kits may include one or more containers comprising present fusion polypeptides, agents, vectors, and/or compositions.

In some embodiments, the kit can comprise instructions for use in any of the methods described herein. The included instructions can comprise a description of administration of the pharmaceutical compositions to a subject to achieve the intended activity in a subject. The kit may further comprise a description of selecting a subject suitable for treatment based on identifying whether the subject is in need of the treatment. In some embodiments, the instructions comprise a description of administering the pharmaceutical composition to a subject who is in need of the treatment.

The instructions relating to the use of the pharmaceutical composition described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

In some embodiment, the disclosure provides articles of manufacture comprising contents of the kits described above.

In some embodiments, the individual components of the formulation can be provided in one container. Alternatively, it can be desirable to provide the components of the formulation separately in two or more containers. The different components can be combined, e.g., according to instructions provided with the kit. The components can be combined according to a method described herein, e.g., to prepare and administer a pharmaceutical composition.

In addition to the the present fusion polypeptide or the present system, the kit can include other ingredients, such as a solvent or buffer, a stabilizer or a preservative, and/or a second agent for treating a condition or disorder.

The present fusion polypeptide, agents, vectors, or compositions can be provided in any form, e.g., liquid, dried or lyophilized form.

### Definitions

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion polypeptide”, “fusion protein”, or “protein chimera” as used herein refers to a hybrid polypeptide which comprises protein domains from at least two different proteins. One domain may be located at the amino-terminal (N-terminal) portion of the fusion protein or at the carboxy-terminal (C-terminal) portion of the fusion protein. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The terms “subject,” “individual,” and “patient” are used interchangeably, and refer to a vertebrate, preferably a mammal such as a human. Mammals include, but are not limited to, human primates, non-human primates or murine, bovine, equine, canine or feline species. In the context of the present disclosure, the term “subject” also encompasses tissues and cells that can be cultured in vitro or ex vivo or manipulated in vivo. The term “subject” can be used interchangeably with the term “organism”.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Examples of polynucleotides include, but are not limited to, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. One or more nucleotides within a polynucleotide can further be modified. The sequence of nucleotides may be interrupted by non-nucleotide components. A polynucleotide may also be modified after polymerization, such as by conjugation with a labeling agent.

The term “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi-stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally-occurring as a whole. Parts of the vectors can be naturally-occurring. The non-naturally occurring recombinant expression vectors of the present disclosure can comprise any type of nucleotides, including, but not limited to DNA and RNA, which can be single-stranded or double-stranded, synthesized or obtained in part from natural sources, and which can contain natural, non-natural or altered nucleotides.

“Transfection,” “transformation,” or “transduction,” as used herein, refer to the introduction of one or more exogenous polynucleotides into a host cell by using physical or chemical methods.

“Antibody,” “fragment of an antibody,” “antibody fragment,” “functional fragment of an antibody,” or “antigen-binding portion” are used interchangeably to mean one or more fragments or portions of an antibody that retain the ability to specifically bind to a specific antigen (Holliger et al., Nat. Biotech. (2005) 23(9): 1126). The present antibodies may be antibodies and/or fragments thereof. Antibody fragments include Fab, F(ab′)2, scFv, disulfide linked Fv, Fc, or variants and/or mixtures. The antibodies may be chimeric, humanized, single chain, or bi-specific. All antibody isotypes are encompassed by the present disclosure, including, IgA, IgD, IgE, IgG, and IgM. Suitable IgG subtypes include IgG1, IgG2, IgG3 and IgG4. An antibody light or heavy chain variable region consists of a framework region interrupted by three hypervariable regions, referred to as complementarity determining regions (CDRs). The CDRs of the present antibodies or antigen-binding portions can be from a non-human or a human source. The framework of the present antibodies or antigen-binding portions can be human, humanized, non-human (e.g., a murine framework modified to decrease antigenicity in humans), or a synthetic framework (e.g., a consensus sequence).

The present antibodies or antigen-binding portions can specifically bind with a dissociation constant (K_{D}) of less than about 10⁻⁷ M, less than about 10⁻⁸ M, less than about 10⁻⁹ M, less than about 10⁻¹⁰ M, less than about 10^{-H} M, or less than about 10⁻¹² M. Affinities of the antibodies according to the present disclosure can be readily determined using conventional techniques (see, e.g., Scatchard et al., Ann. N.Y. Acad. Sci. (1949) 51:660; and U.S. Pat. Nos. 5,283,173, 5,468,614, or the equivalent).

The antigen recognition moiety of the fusion protein encoded by the nucleic acid sequence can contain any lineage antigen-specific, antigen-binding antibody fragment. The antibody fragment can comprise one or more CDRs, the variable region (or portions thereof), the constant region (or portions thereof), or combinations of any of the foregoing.

The term “cell lineage” refers to cells with a common ancestry and developing from the same type of identifiable cell into specific identifiable/functioning cells. The cell lineages used herein include, but are not limited to, respiratory, prostatic, pancreatic, mammary, renal, intestinal, neural, skeletal, vascular, hepatic, hematopoietic, muscle or cardiac cell lineages.

The term “inhibition” when used in reference to gene expression or function of a lineage specific antigen refers to a decrease in the level of gene expression or function of the lineage specific antigen, where the inhibition is a result of interference with gene expression or function. The inhibition may be complete, in which case there is no detectable expression or function, or it may be partial. Partial inhibition can range from near complete inhibition to a near absence of inhibition.

The terms “treat”, “treatment”, and the like refer to a means to slow down, relieve, ameliorate or alleviate at least one of the symptoms of the disease, or reverse the disease after its onset.

“Treating” or “treatment” of a state, disorder or condition includes:
(1) preventing or delaying the appearance of clinical symptoms of the state, disorder, or condition developing in a person who may be afflicted with or predisposed to the state, disorder or condition but does not yet experience or display clinical symptoms of the state, disorder or condition; or
(2) inhibiting the state, disorder or condition, i.e., arresting, reducing or delaying the development of the disease or a relapse thereof (in case of maintenance treatment) or at least one clinical symptom, sign, or test, thereof; or
(3) relieving the disease, i.e., causing regression of the state, disorder or condition or at least one of its clinical or sub-clinical symptoms or signs.

The benefit to a subject to be treated is either statistically significant or at least perceptible to the patient or to the physician.

The terms “prevent”, “prevention”, and the like refer to acting prior to overt disease onset, to prevent the disease from developing or minimize the extent of the disease or slow its course of development.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

An “immune response” refers to the development in the host of a cellular and/or antibody-mediated immune response to a composition or vaccine of interest. Such a response usually consists of the subject producing antibodies, B cells, helper T cells, suppressor T cells, regulatory T cells, and/or cytotoxic T cells directed specifically to an antigen or antigens included in the composition or vaccine of interest.

A “therapeutically effective amount” means the amount of a compound that, when administered to an animal for treating a state, disorder or condition, is sufficient to effect such treatment. The “therapeutically effective amount” will vary depending on the compound, the disease and its severity and the age, weight, physical condition and responsiveness of the animal to be treated.

The compositions of the invention may include a “therapeutically effective amount” or a “prophylactically effective amount” of a compound described herein. A “therapeutically effective amount” refers to an amount effective, at dosages and for periods of time necessary, to achieve the desired therapeutic result. A therapeutically effective amount of an antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

While it is possible to use a composition provided by the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent and/or carrier. The excipient, diluent and/or carrier must be “acceptable” in the sense of being compatible with the other ingredients of the formulation and not deleterious to the recipient thereof.

The compositions of the invention can be formulated for administration in any convenient way for use in human or veterinary medicine. The invention therefore includes within its scope pharmaceutical compositions comprising a product of the present invention that is adapted for use in human or veterinary medicine.

In one embodiment, the pharmaceutical composition is administered as an oral formulation. Oral dosage forms are well known in the art and include tablets, caplets, gelcaps, capsules, and medical foods. Tablets, for example, can be made by well-known compression techniques using wet, dry, or fluidized bed granulation methods.

Such oral formulations may be presented for use in a conventional manner with the aid of one or more suitable excipients, diluents, and carriers. Pharmaceutically acceptable excipients assist or make possible the formation of a dosage form for a bioactive material and include diluents, binding agents, lubricants, glidants, disintegrants, coloring agents, and other ingredients. Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used. An excipient is pharmaceutically acceptable if, in addition to performing its desired function, it is non-toxic, well tolerated upon ingestion, and does not interfere with absorption of bioactive materials.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

The dosage of the therapeutic formulation will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some cases, oral administration will require a higher dose than if administered intravenously. In some cases, topical administration will include application several times a day, as needed, for a number of days or weeks in order to provide an effective topical dose.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, olive oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “agent” as used herein means a substance that produces or is capable of producing an effect and would include, but is not limited to, chemicals, pharmaceuticals, biologics, small organic molecules, antibodies, nucleic acids, peptides, and proteins.

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include, but are not limited to, plasmids, phages, and viruses. Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA which codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct” or “gene construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described herein.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “homologous,” as used herein is an art-understood term that refers to nucleic acids or polypeptides that are highly related at the level of nucleotide and/or amino acid sequence. Nucleic acids or polypeptides that are homologous to each other are termed “homologs.” Homology between two sequences can be determined by sequence alignment methods known to those of skill in the art. In accordance with the invention, two sequences are considered to be homologous if they are at least about 50-60% identical, e.g., share identical residues (e.g., amino acid residues) in at least about 50-60% of all residues comprised in one or the other sequence, at least about 70% identical, at least about 80% identical, at least about 90% identical, at least about 95% identical, at least about 98% identical, at least about 99% identical, at least about 99.5% identical, or at least about 99.9% identical, for at least one stretch of at least 20, at least 30, at least 40, at least 50, at least 60, at least 70, at least 80, at least 90, at least 100, at least 120, at least 150, or at least 200 amino acids.

The term “linker,” as used herein, refers to a chemical group or a molecule linking two adjacent molecules or moieties, e.g., an antigen-binding fragment that binds a lineage-specific cell-surface antigen, and a polypeptide that binds a molecule expressed on natural killer (NK) cells. In some embodiments, a linker joins a V_{H} and a V_{L} (e.g., of an anti-CD33 antibody fragment). In some embodiments, a linker joins a V_{L} and a polypeptide that binds a molecule expressed on natural killer (NK) cells (e.g., an ectodomain of ULBP1, ULBP2, ULBP3, ULBP4, ULBP5, ULBP6, MICA, MICB, or HCMV UL18). In one embodiment, the linker is positioned between, or flanked by, two groups, molecules, or other moieties and connected to each one via a covalent bond, thus connecting the two. In some embodiments, the linker is an amino acid or a plurality of amino acids (a peptide linker). In some embodiments, the linker is an organic molecule, group, polymer, or chemical moiety (a non-peptide linker). In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “pharmaceutical composition,” as used herein, refers to a composition that can be administrated to a subject in the context of treatment and/or prevention of a disease or disorder. In some embodiments, a pharmaceutical composition comprises an active ingredient, e.g., the present fusion polypeptide, nucleic acid molecule, vector, agent, etc., and optionally a pharmaceutically acceptable excipient.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1 Expression and Purification of αCD33-ULBP1 Chimera Proteins

Expression of anti-CD33-ULBP1 chimeras in cells was assayed. Briefly, 293T cells were mock transfected (no plasmid) or transfected with anti-CD33-ULBP1 (αCD33-ULBP1) chimeras 1 and 2 plasmids. The cell pellets were lysed in 2× SDS gel loading buffer. The lysates were loaded on an SDS-PAGE gel. The separated proteins were transferred to a nylon membrane. The membrane was probed with ant-MYC antibody to detect CD33-ULBP1 and anti-beta-actin to detect the actin protein (loading control). The membrane was also probed with a fluorochrome-conjugated secondary antibody to detect the primary antibody.

Expression and purification of anti-CD33-ULBP1 chimeras in cell culture supernatant were assayed. Briefly, cell culture supernatant of 293T cells mock transfected (no plasmid) or transfected with anti-CD33-ULBP1 chimeras 1 or 2 plasmids were subjected to affinity purification using Talon beads. The input, flow-through, and the purified protein (elute) were then separated on an SDS-PAGE gel and transferred to a nylon membrane. The membrane was first probed with anti-MYC antibodies, and then a fluorochrome-conjugated secondary antibody to detect the primary antibody.

### Methods

### Anti-CD33-ULBP1 Chimera:

The chimera protein was assembled as follows: IL2 secretory signal sequence followed by anti-CD33 ScFv, a linker (with or without an additional proline), ULBP1 ectodomain, a MYC epitope tag, and a 6× histidine. The IL2 signal sequence enables protein to be secreted in the cell culture supernatant. The anti-CD33 ScFv domain binds the CD33 antigen expressing cells. The linker is used to link the anti-CD33 ScFv domain and the ULBP1 ectodomain. In one variation, a linker with an additional proline is used to modify orientation. ULBP1 ectodomain binds the NKG2D-expressing cells (e.g., NK cells). The Myc and 6× histidine epitope tags are used for tandem immuno- and affinity purification. The assembled fusion protein chimera sequence was reverse translated to obtain the DNA sequence which was further modified to obtain a codon-optimized sequence.

### Plasmid Construct:

Plasmid constructs expressing anti-CD33-ULBP1 chimera were made by cloning a synthesized DNA fragment in mammalian expression vector pcDNA3.4 under a CMV promoter. Plasmid DNA was amplified in, and purified from, E. Coli using standard procedures.

### Expression:

anti-CD33-ULBP1 chimera was expressed in 293T cells. Plasmid constructs expressing anti-CD33-ULBP1 were transiently transfected into 239T cells which were grown at 37° C. and 5% CO₂ in a humidified chamber. After 72 hours, cell culture supernatant containing the secreted anti-CD33-ULBP1 chimera was collected and centrifuged.

### Purification:

The anti-CD33-ULBP1 chimera protein was purified from the cell culture supernatant using Talon beads (Cobalt). Briefly, 10 uL Talon beads per milliliter of the supernatant, containing 1 mM PMSF, was incubated for 2-3 hours on a rotating platform at 4° C. After rotation, the beads were collected using centrifugation, washed twice with PBS, and the protein was eluted using either imidazole or EDTA.

### Immunoblot:

The expression level of the anti-CD33-ULBP1 chimera and the purity of the purified product were assayed using SDS-PAGE and immunoblotting using anti-MYC antibodies following standard procedure.

### Example 2 Binding Assay of αCD33-ULBP1 Chimera Proteins on NK Primary Cells and CD34+ Cells

The binding of the anti-CD33-ULBP1 chimera with CD33 antigen-expressing cord blood CD34+ cells or NKG2D-expressing primary NK cells was tested using flow cytometry. Briefly, primary NK cells were purified from peripheral blood of donors. CD34+ cells were purified from cord blood of donors. Purified cells were incubated with the elute from the mock, chimera 1 or chimera 2 proteins purified from the cell supernatant. The cells were also incubated with FITC-conjugated anti-Myc antibody and analyzed using a flow cytometer.

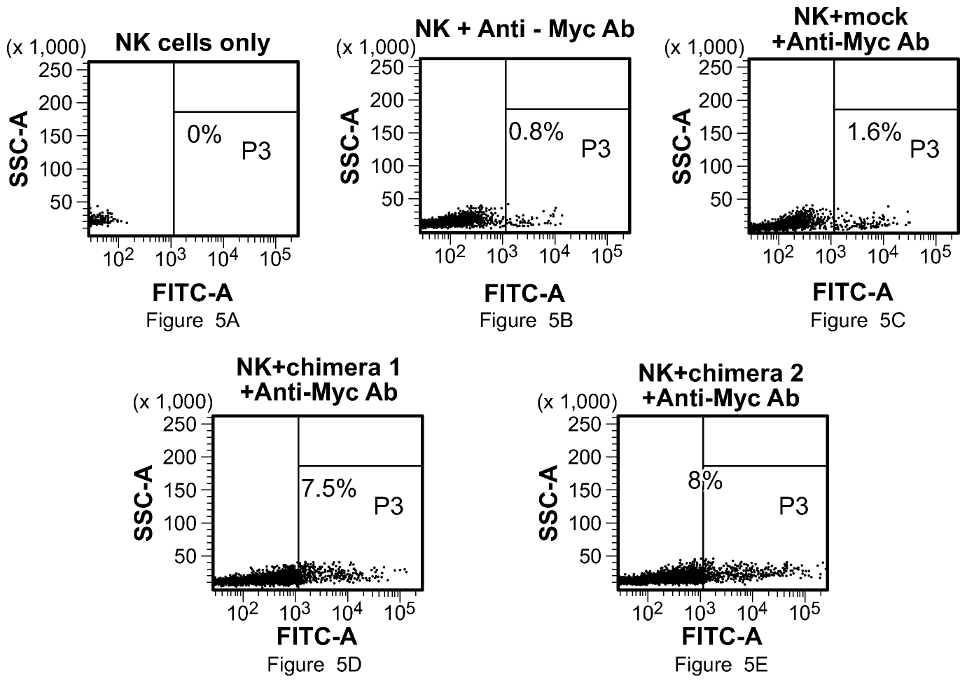

FIGS. 5A-5E are flow cytometry dot plots showing specific binding of chimera 1 and 2 with primary NK cells. As expected, a higher binding signal was observed when protein chimera was present (FIGS. 5D and 5E) but not in controls (FIGS. 5A-5C).

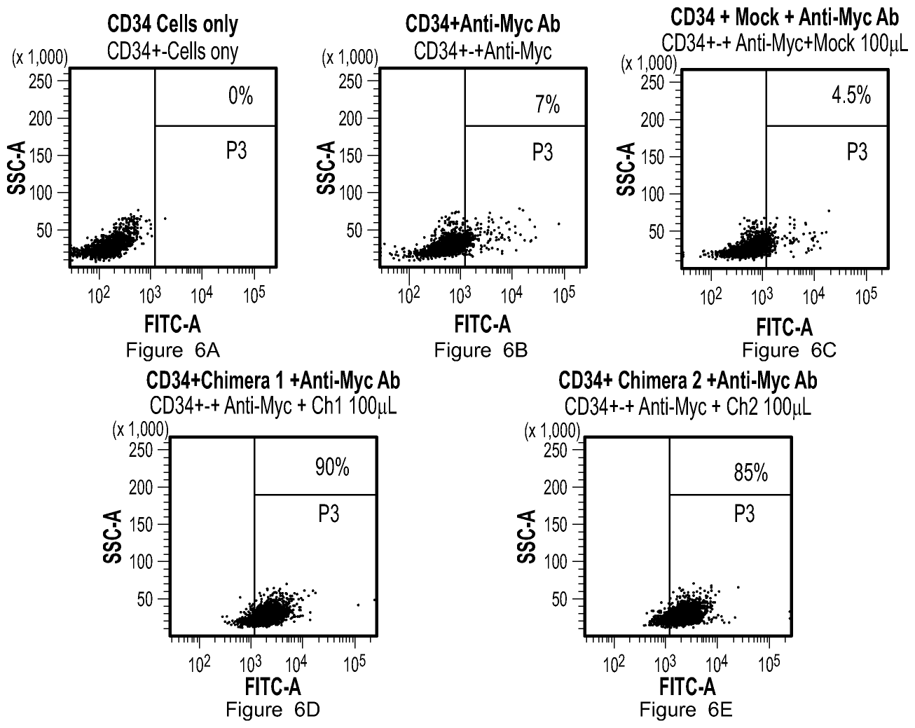

FIGS. 6A-6E are flow cytometry dot plots showing specific binding of chimera 1 and 2 with CD34+ cells. As expected, a higher binding signal was observed when protein chimera was present (FIGS. 6D and 6E) but not in controls (FIGS. 6A-6C).

### Example 3 Functional Assays of αCD33-ULBP1 Chimera Proteins

### In Vitro Cytotoxicity Assays:

To assay functional activity of the anti-CD33-ULBP1 chimera, we will perform cytotoxicity assays. Cells expressing CD33 (HL-60, K562, or primary CD34+ cells from normal and cancer patients) will be stained with Celltrace blue and co-incubated with or without anti-CD33-ULBP1 chimera at various concentrations and various effector to target ratio of primary NK cells for 16 to 24 hours. Cell death will measure using 7AAD viability dye and specific target lysis will be determined.

### In Vitro Cytokine Production and Degranulation Assay:

To assay for NK cell activation, cells expressing CD33 (HL-60, K562, or primary CD34⁺ cells from normal and cancer patients) will be co-incubated with or without anti-CD33-ULBP1 chimera and primary NK cells for 24 hours. CD107a expression and intracellular IFN-γ and TNF-α production will be measured using flow cytometry.

### In Vivo Assay:

NSG-SGM3 mice will be conditioned with sublethal (1.2 Gy) total-body irradiation and 5×105 HL-60 cells expressing either luciferase or dTomato will be injected intravenously into the mice within 12-hours post-irradiation. Two weeks later, mice will be treated with 2×106 NK cells with or without 5 ug of anti-CD33-ULBP1 or PBS intravenously injected. Mice will be monitored daily with a weekly image using fluorescent imaging using the PerkinElmer IVIS Spectrum Optical Imaging System. Images will be acquired and analyzed with Living Image 4.4 Optical Imaging Analysis Software.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Sequences:
NKG2D-binding domain of human UL16-binding
protein 1 (ULBP1), ULBP1 ectodomain (27 to
216 aa of Q9BZM6-1) (SEQ ID NO: 1):
WVDTHCLCYDFIITPKSRPEPQWCEVQGLVDERPFLHYDCVNHKAK
AFASLGKKVNVTKTWEEQTETLRDVVDFLKGQLLDIQVENLIPIEP
LTLQARMSCEHEAHGHGRGSWQFLFNGQKFLLFDSNNRKWTALHPG
AKKMTEKWEKNRDVTMFFQKISLGDCKMWLEEFLMYWEQMLDPTKP
PSLAPG
NKG2D-binding domain of human UL16-binding
protein 2 (ULBP2) (SEQ ID NO: 2):
GRADPHSLCYDITVIPKFRPGPRWCAVQGQVDEKTFLHYDCGNKTV
TPVSPLGKKLNVTTAWKAQNPVLREVVDILTEQLRDIQLENYTPKE
PLTLQARMSCEQKAEGHSSGSWQFSFDGQIFLLFDSEKRMWTTVHP
GARKMKEKWENDKVVAMSFHYFSMGDCIGWLEDFLMGMDSTLEPSA
GAPLAMSS
NKG2D-binding domain of human UL16-binding
protein 3 (ULBP3) (SEQ ID NO: 3):
DAHSLWYNFTIIHLPRHGQQWCEVQSQVDQKNFLSYDCGSDKVLSM
GHLEEQLYATDAWGKQLEMLREVGQRLRLELADTELEDFTPSGPLT
LQVRMSCECEADGYIRGSWQFSFDGRKFLLFDSNNRKWTVVHAGAR
RMKEKWEKDSGLTTFFKMVSMRDCKSWLRDFLMHRKKRLEPTAPPT
MAPG
NKG2D-binding domain of human Retinoic acid
early transcript 1E (ULBP4, RAET1E) (SEQ ID
NO: 4):
HSLCFNFTIKSLSRPGQPWCEAQVFLNKNLFLQYNSDNNMVKPLGL
LGKKVYATSTWGELTQTLGEVGRDLRMLLCDIKPQIKTSDPSTLQV
EMFCQREAERCTGASWQFATNGEKSLLFDAMNMTWTVINHEASKIK
ETWKKDRGLEKYFRKLSKGDCDHWLREFLGHWEAMPEPTVSPVNAS
DIHWSSSSLPD
NKG2D-binding domain of human UL-16 binding
protein 5 (ULBP5, RAET1G) (SEQ ID NO: 5):
GLADPHSLCYDITVIPKFRPGPRWCAVQGQVDEKTFLHYDCGSKTV
TPVSPLGKKLNVTTAWKAQNPVLREVVDILTEQLLDIQLENYIPKE
PLTLQARMSCEQKAEGHGSGSWQLSFDGQIFLLFDSENRMWTTVHP
GARKMKEKWENDKDMTMSFHYISMGDCTGWLEDFLMGMDSTLEPSA
GAPPTMSSGTAQPR
NKG2D-binding domain of human UL16-binding
protein 6 (ULBP6, RAET1L) (SEQ ID NO: 6):
RRDDPHSLCYDITVIPKFRPGPRWCAVQGQVDEKTFLHYDCGNKTV
TPVSPLGKKLNVTMAWKAQNPVLREVVDILTEQLLDIQLENYTPKE
PLTLQARMSCEQKAEGHSSGSWQFSIDGQTFLLFDSEKRMWTTVHP
GARKMKEKWENDKDVAMSFHYISMGDCIGWLEDFLMGMDSTLEPSA
GAPLAMSSG
NKG2D-binding domain of human MHC class I
polypeptide-related sequence A (MICA) (SEQ ID
NO: 7):
EPHSLRYNLTVLSWDGSVQSGFLTEVHLDGQPFLRCDRQKCRAKPQ
GQWAEDVLGNKTWDRETRDLTGNGKDLRMTLAHIKDQKEGLHSLQE
IRVCEIHEDNSTRSSQHFYYDGELFLSQNLETKEWTMPQSSRAQTL
AMNVRNFLKEDAMKTKTHYHAMHADCLQELRRYLKSGVVLRRTVPP
MVNVTRSEASEGNITVTCRASGFYPWNITLSWRQDGVSLSHDTQQW
GDVLPDGNGTYQTWVATRICQGEEQRFTCYMEHSGNHSTHPVPSGK
VLVLQSHW
NKG2D-binding domain of human MHC class I
polypeptide-related sequence B (MICB) (SEQ ID
NO: 8):
AEPHSLRYNLMVLSQDESVQSGFLAEGHLDGQPFLRYDRQKRRAKP
QGQWAEDVLGAKTWDTETEDLTENGQDLRRTLTHIKDQKGGLHSLQ
EIRVCEIHEDSSTRGSRHFYYDGELFLSQNLETQESTVPQSSRAQT
LAMNVTNFWKEDAMKTKTHYRAMQADCLQKLQRYLKSGVAIRRTVP
PMVNVTCSEVSEGNITVTCRASSFYPRNITLTWRQDGVSLSHNTQQ
WGDVLPDGNGTYQTWVATRIRQGEEQRFTCYMEHSGNHGTHPVPSG
KVLVLQSQRTD
NKG2D-binding domain of Raet1a (SEQ ID NO: 29):
LDDAHSLRCNLTIKDPTPADPLWYEAKCLVDEILILHLSNINKTMT
SGDPGETANATEVGECLTQPLKDLCQKLRNKVSNTKVDTHKTNGYP
HLQVTMIYLQSQGQIPSATWEFNISDSYFFTFYTENMSWRSANDES
GVIMNKWKDDGEFVKRLKFLIPECRQEVDEFLKQPKEKPRSTSRSP
SITQLTSTSPLPPPSHS
(species: mouse)
NKG2D-binding domain of Raet1b (SEQ ID NO: 30):
LDDAHSLRCNLTIKDPTPADPLWYEAKCFVGEILILHLSNINKTMT
SGDPGETANATEVKKCLTQPLKNLCQKLRNKVSNTKVDTHKTNGYP
HLQVTMIYPQSQGRTPSATWEFNISDSYFFTFYTENMSWRSANDES
GVIMNKWKDDGEFVKQLKFLIHECSQKMDEFLKQSKEKPRSTSRSP
SITQLTSTSPLPPPSHS
(species: mouse)
NKG2D-binding domain of Raet1c (SEQ ID NO: 31):
LDDAHSLRCNLTIKAPTPADPLWYEAKCLVDEILILHLSNINKTMT
SGDPGETANATEVGECLTQPVNDLCQKLRDKVSNTKVDTHKTNGYP
HLQVTMIYPQSQGQTPSATWEFNISDSYFFTFYTENMSWRSANDES
GVIMNKWNDDGDLVQRLKYFIPECRQKIDEFLKQSKEKPRSTSRSP
SITQLTSTSPLPPPS
(species: mouse)
NKG2D-binding domain of Retinoic acid early-
inducible protein 1-delta (RAET1D) (SEQ ID
NO: 9):
LDDAHSLRCNLTIKAPTPADEVKCFVGEILILHLSNINKTMTSGDP
GETANATEVGECLTQPLKDLCQKLRDKVSNTKVDTHKTNGYPHLQV
TMIYPQSQGQTPSATWEFNISDSYFFTFYTEIMSWRSANDESGVIM
NKWKDDGEFVKQLKFLIHGCSQKMDEFLKQSKEKPRSTSRSPSITQ
LTSTSPLPPTSHS
(species: mouse)
NKG2D-binding domain of Raet1e (SEQ ID NO: 32):
LDDAHSLRCNLTIKDPTSADLPWCDVKCSVDEITILHLNNINKTMT
SGDPGKMANATGKCLTQPLNDLCQELRDKVSNTKVDTHKTNGYPHL
QVTMIYPQSQGQTPSATWEFNISDSYFFTFYTENMSWRSANDESGV
IMNKWKDDGDLVQQLKYFIPQCRQKIDEFLKQSKEKPRSTSRSPSI
TQLTSTSPLPPPS
(species: mouse)
NKG2D-binding domain of H60b (SEQ ID NO: 33):
TGTDSLSCELTFNHRTLHGQCSVNGKTLLDFGDKKHEGNATEMCAD
LSQSLRELSEGMRNQQSGNDALNVTTQSQYNQGEFIGGFWAINTDE
QHSIYFYPLNMTWRESHSDNSSAMEHWKNKNLEKDIRNVLIIYFSR
CLNKLSPHFREMPKSKIKVLDTTQNTNTTQIHPTVNNSQHNSDTQG
LSFTWIVIICIGGIVSFMAFMVFAWCMLKKKKGALCCSSSSTT
(species: mouse)
NKG2D-binding domain of H60c (SEQ ID NO: 34):
LNCKLTVKYRTLQGLCSVNGKTFLDFGDENHEGNATMLCPALYQSL
TDISEVMWSLQSGNDALNVTTRSQYYQGEFIDGFWDINTDEQHSIY
VYPLNKTWRESHSDNSSAMEQWKNKNLEKDIRNVLMVDFSCCLNKS
SPHFREMPTLPTTAAHVDQPRS
(species: mouse)
Anti-CD33 Light Chain Variable Region (V_{L})-
amino acid sequence (SEQ ID NO: 10)
(CDR1-3 bolded and underlined):
EIVLTQSPGSLAVSPGERVTMSCKSSQSVFFSSSQKNYLAWYQQIP
GQSPRLLIYWASTRESGVPDRFTGSGSGTDFTLTISSVQPEDLAIY
YCHQYLSSRTFGQGTKLEIKR
(species: synthetic)
CDR1 of anti-CD33 Light Chain Variable Region
(V_{L}) (SEQ ID NO: 11):
KSSQSVFFSSSQKNYLA
(species: mouse)
CDR2 of anti-CD33 Light Chain Variable Region
(V_{L}) (SEQ ID NO: 12):
WASTRES
(species: mouse)
CDR3 of anti-CD33 Light Chain Variable Region
(V_{L}) (SEQ ID NO: 13):
HQYLSSRT
(species: mouse)
Anti-CD33 Heavy Chain Variable Region (V_{L})-
nucleic acid sequence (SEQ ID NO: 14):
GAGATCGTGCTGACCCAGAGCCCCGGCAGCCTGGCCGTGAGCCCCG
GCGAGAGGGTGACCATGAGCTGCAAGAGCAGCCAGAGCGTGTTCTT
CAGCAGCAGCCAGAAGAACTACCTGGCCTGGTACCAGCAGATCCCC
GGCCAGAGCCCCAGGCTGCTGATCTACTGGGCCAGCACCAGGGAGA
GCGGCGTGCCCGACAGGTTCACCGGCAGCGGCAGCGGCACCGACTT
CACCCTGACCATCAGCAGCGTGCAGCCCGAGGACCTGGCCATCTAC
TACTGCCACCAGTACCTGAGCAGCAGGACCTTCGGCCAGGGCACCA
AGCTGGAGATCAAGAGG
(species: synthetic)
Anti-CD33 Heavy Chain Variable Region (V_{H})-
amino acid sequence (SEQ ID NO: 15)
(CDR1-3 bolded and underlined):
QVQLQQPGAEVVKPGASVKMSCKASGYTFTSYYIHWIKQTPGQGLE
WVGVIYPGNDDISYNQKFQGKATLTADKSSTTAYMQLSSLTSEDSA
VYYCAREVRLRYFDVWGQGTTVTVSS
(species: synthetic)
CDR1 of Anti-CD33 Heavy Chain Variable Region
(V_{H}) (SEQ ID NO: 16):
SYYIH
(species: mouse)
CDR2 of Anti-CD33 Heavy Chain Variable Region
(V_{H}) (SEQ ID NO: 17):
VIYPGNDDISYNQKFQG
(species: mouse)
CDR3 of Anti-CD33 Heavy Chain Variable Region
(V_{H}) (SEQ ID NO: 18):
EVRLRYFDV
(species: mouse)
Anti-CD33 Heavy Chain Variable Region (V_{H})-
nucleic acid sequence (SEQ ID NO: 19):
CAGGTGCAGCTGCAGCAGCCCGGCGCCGAGGTGGTGAAGCCCGGCG
CCAGCGTGAAGATGAGCTGCAAGGCCAGCGGCTACACCTTCACCAG
CTACTACATCCACTGGATCAAGCAGACCCCCGGCCAGGGCCTGGAG
TGGGTGGGCGTGATCTACCCCGGCAACGACGACATCAGCTACAACC
AGAAGTTCCAGGGCAAGGCCACCCTGACCGCCGACAAGAGCAGCAC
CACCGCCTACATGCAGCTGAGCAGCCTGACCAGCGAGGACAGCGCC
GTGTACTACTGCGCCAGGGAGGTGAGGCTGAGGTACTTCGACGTGT
GGGGCCAGGGCACCACCGTGACCGTGAGCAGC
(species: synthetic)
CD33-ULBP1-1 (SEQ ID NO: 20):
MYRMQLLSCIALSLALVTNSEIVLTQSPGSLAVSPGERVTMSCKSS
QSVFFSSSQKNYLAWYQQIPGQSPRLLIYWASTRESGVPDRFTGSG
SGTDFTLTISSVQPEDLAIYYCHQYLSSRTFGQGTKLEIKRGSTSG
SGKPGSGEGSTKGQVQLQQPGAEVVKPGASVKMSCKASGYTFTSYY
IHWIKQTPGQGLEWVGVIYPGNDDISYNQKFQGKATLTADKSSTTA
YMQLSSLTSEDSAVYYCAREVRLRYFDVWGQGTTVTVSSSSSAGGG
GSGGGGSGGGGS

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

EQKLISE
EDLHHHHHH
(species: synthetic)
CD33-ULBP1-2 (With proline) (SEQ ID NO: 21):
MYRMQLLSCIALSLALVTNSEIVLTQSPGSLAVSPGERVTMSCKSS
QSVFFSSSQKNYLAWYQQIPGQSPRLLIYWASTRESGVPDRFTGSG
SGTDFTLTISSVQPEDLAIYYCHQYLSSRTFGQGTKLEIKRGSTSG
SGKPGSGEGSTKGQVQLQQPGAEVVKPGASVKMSCKASGYTFTSYY
IHWIKQTPGQGLEWVGVIYPGNDDISYNQKFQGKATLTADKSSTTA
YMQLSSLTSEDSAVYYCAREVRLRYFDVWGQGTTVTVSSSSSAGGG
GSGGGGSGGGGSP

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

EQKLISEEDLHHHHHH
(species: synthetic)
ULBP1-CD33-1 (SEQ ID NO: 22):
MYRMQLLSCIALSLALVTNSWVDTHCLCYDFIITPKSRPEPQWCEV
QGLVDERPFLHYDCVNHKAKAFASLGKKVNVTKTWEEQTETLRDVV
DFLKGQLLDIQVENLIPIEPLTLQARMSCEHEAHGHGRGSWQFLFN
GQKFLLFDSNNRKWTALHPGAKKMTEKWEKNRDVTMFFQKISLGDC
KMWLEEFLMYWEQMLDPTKPPSLAPGTTQPSSSAGGGGSGGGGSGG
GGSEIVLTQSPGSLAVSPGERVTMSCKSSQSVFFSSSQKNYLAWYQ
QIPGQSPRLLIYWASTRESGVPDRFTGSGSGTDFTLTISSVQPEDL
AIYYCHQYLSSRTFGQGTKLEIKRGSTSGSGKPGSGEGSTKGQVQL
QQPGAEVVKPGASVKMSCKASGYTFTSYYIHWIKQTPGQGLEWVGV
IYPGNDDISYNQKFQGKATLTADKSSTTAYMQLSSLTSEDSAVYYC
AREVRLRYFDVWGQGTTVTVSS [symbol] HHHHHH
(species: synthetic)
IL2 secretory signal sequence (SEQ ID NO: 23):
MYRMQLLSCIALSLALVTNS
(species: human)
Linker_1 (SEQ ID NO: 24):
GSTSGSGKPGSGEGSTKG
(species: synthetic)
Linker_2-(GGGGS) × 3 (SEQ ID NO: 25):
SSSAGGGGSGGGGSGGGGS
(species: synthetic)
Linker_2P-(GGGGS) × 3 (SEQ ID NO: 26)
SSSAGGGGSGGGGSGGGGSP
(species: synthetic)
Myc tag (SEQ ID NO: 27):
EQKLISEEDL
(species: human)
His6 tag (SEQ ID NO: 28):
HHHHHH
(species: synthetic)

1. A fusion polypeptide, comprising:
(i) an antigen-binding fragment that binds a lineage-specific cell-surface antigen; and
(ii) a polypeptide that binds a molecule expressed on natural killer (NK) cells.

2. The fusion polypeptide of claim 1, wherein the molecule expressed on NK cells is a ligand or receptor expressed on NK cells.

3. The fusion polypeptide of claim 1, wherein polypeptide that binds the molecule expressed on NK cells is a ligand for a NK cell surface receptor.

4. The fusion polypeptide of claim 1, wherein the molecule expressed on NK cells is NKG2D, CD16, or CD2.

5. The fusion polypeptide of claim 1, wherein the polypeptide that binds a molecule expressed on NK cells is ULBP1, ULBP2, ULBP3, ULBP4, ULBP5, ULBP6, MICA, MICB, or mutants or fragments thereof.

6. The fusion polypeptide of claim 1, wherein the polypeptide that binds a molecule expressed on NK cells is an ectodomain of ULBP1, ULBP2, ULBP3, ULBP4, ULBP5, ULBP6, MICA, or MICB.

7. The fusion polypeptide of claim 1, wherein the lineage-specific cell-surface antigen is CD33.

8. The fusion polypeptide of claim 1, wherein the antigen-binding fragment is a single-chain antibody fragment (scFv).

9. The fusion polypeptide of claim 1, wherein the antigen-binding fragment comprises an amino acid sequence at least 90% identical to the amino acid sequence set forth in SEQ ID NO: 10.

10. The fusion polypeptide of claim 1, wherein the antigen-binding fragment comprises an amino acid sequence at least 90% identical to the amino acid sequence set forth in SEQ ID NO: 15.

11. The fusion polypeptide of claim 1, wherein the antigen-binding fragment comprises a heavy chain variable region comprising complementary determining regions (CDRs) comprising amino acid sequences at least 90% identical to the amino acid sequences set forth in SEQ ID NO: 16, SEQ ID NO: 17, and SEQ ID NO: 18, respectively.

12. The fusion polypeptide of claim 1, wherein the antigen-binding fragment comprises a light chain variable region comprising complementary determining regions (CDRs) comprising amino acid sequences at least 90% identical to the amino acid sequences set forth in SEQ ID NO: 11, SEQ ID NO: 12, and SEQ ID NO: 13, respectively.

13. The fusion polypeptide of claim 1, comprising an amino acid sequence at least 90% identical to the amino acid sequence set forth in SEQ ID NO: 20, SEQ ID NO: 21, or SEQ ID NO: 22.

14. A composition comprising the fusion polypeptide of claim 1.

15. A nucleic acid molecule encoding the fusion polypeptide of claim 1.

16. (canceled)

17. A vector comprising the nucleic acid molecule of claim 15.

18. (canceled)

19. A cell comprising the vector of claim 17.

20. (canceled)

21. A composition comprising:
(i) a first antigen-binding fragment that binds a lineage-specific cell-surface antigen; and
(ii) a second polypeptide that binds a molecule expressed on natural killer (NK) cells,
wherein the first antigen-binding fragment comprises a first dimerization motif, wherein the second polypeptide comprises a second dimerization motif, and wherein the first dimerization motif binds to the second dimerization motif.

22.-26. (canceled)

27. A composition comprising at least one vector encoding:
(i) a first antigen-binding fragment that binds a lineage-specific cell-surface antigen; and
(ii) a second polypeptide that binds a molecule expressed on natural killer (NK) cells, wherein the first antigen-binding fragment comprises a first dimerization motif, wherein the second polypeptide comprises a second dimerization motif, and wherein the first dimerization motif binds to the second dimerization motif.

28. (canceled)

29. A method of treating a hematopoietic malignancy in a subject, comprising administering to the subject an effective amount of the composition of claim 14.

30. The method of claim 29, wherein the hematopoietic malignancy is a myeloid malignancy, Hodgkin's lymphoma, non-Hodgkin's lymphoma, leukemia, multiple myeloma acute myeloid leukemia, chronic myelogenous leukemia, acute lymphoblastic leukemia, or chronic lymphoblastic leukemia.

31. (canceled)

32. (canceled)

33. The fusion polypeptide of claim 1, wherein the first antigen-binding fragment comprises a first dimerization motif, the second polypeptide comprises a second dimerization motif, and the first dimerization motif binds to the second dimerization motif.


SEQUENCE LISTING (34 sequences)

SEQ ID NO: 1 - protein, 190 aa, Human
WVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPG

SEQ ID NO: 2 - protein, 192 aa, Human
GRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSS

SEQ ID NO: 3 - protein, 188 aa, Human
DAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPG

SEQ ID NO: 4 - protein, 195 aa, Human
HSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPD

SEQ ID NO: 5 - protein, 198 aa, Human
GLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPR

SEQ ID NO: 6 - protein, 193 aa, Human
RRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSG

SEQ ID NO: 7 - protein, 284 aa, Human
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 8 - protein, 287 aa, Human
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTD

SEQ ID NO: 9 - protein, 197 aa, Murine
LDDAHSLRCNLTIKAPTPADEVKCFVGEILILHLSNINKTMTSGDPGETANATEVGECLTQPLKDLCQKLRDKVSNTKVDTHKTNGYPHLQVTMIYPQSQGQTPSATWEFNISDSYFFTFYTEIMSWRSANDESGVIMNKWKDDGEFVKQLKFLIHGCSQKMDEFLKQSKEKPRSTSRSPSITQLTSTSPLPPTSHS

SEQ ID NO: 10 - protein, 113 aa, Artificial Sequence
  Synthetic
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 11 - protein, 17 aa, Murine
KSSQSVFFSSSQKNYLA

SEQ ID NO: 12 - protein, 7 aa, Murine
WASTRES

SEQ ID NO: 13 - protein, 8 aa, Murine
HQYLSSRT

SEQ ID NO: 14 - DNA, 339 bp, Artificial Sequence
  Synthetic
gagatcgtgctgacccagagccccggcagcctggccgtgagccccggcgagagggtgaccatgagctgcaagagcagccagagcgtgttcttcagcagcagccagaagaactacctggcctggtaccagcagatccccggccagagccccaggctgctgatctactgggccagcaccagggagagcggcgtgcccgacaggttcaccggcagcggcagcggcaccgacttcaccctgaccatcagcagcgtgcagcccgaggacctggccatctactactgccaccagtacctgagcagcaggaccttcggccagggcaccaagctggagatcaagagg

SEQ ID NO: 15 - protein, 118 aa, Artificial Sequence
  Synthetic
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 16 - protein, 5 aa, Murine
SYYIH

SEQ ID NO: 17 - protein, 17 aa, Murine
VIYPGNDDISYNQKFQG

SEQ ID NO: 18 - protein, 9 aa, Murine
EVRLRYFDV

SEQ ID NO: 19 - DNA, 354 bp, Artificial Sequence
  Synthetic
caggtgcagctgcagcagcccggcgccgaggtggtgaagcccggcgccagcgtgaagatgagctgcaaggccagcggctacaccttcaccagctactacatccactggatcaagcagacccccggccagggcctggagtgggtgggcgtgatctaccccggcaacgacgacatcagctacaaccagaagttccagggcaaggccaccctgaccgccgacaagagcagcaccaccgcctacatgcagctgagcagcctgaccagcgaggacagcgccgtgtactactgcgccagggaggtgaggctgaggtacttcgacgtgtggggccagggcaccaccgtgaccgtgagcagc

SEQ ID NO: 20 - protein, 494 aa, Artificial Sequence
  Synthetic
MYRMQLLSCIALSLALVTNSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSSSAGGGGSGGGGSGGGGSWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGEQKLISEEDLHHHHHH

SEQ ID NO: 21 - protein, 495 aa, Artificial Sequence
  Synthetic
MYRMQLLSCIALSLALVTNSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSSSAGGGGSGGGGSGGGGSPWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGEQKLISEEDLHHHHHH

SEQ ID NO: 22 - protein, 498 aa, Artificial Sequence
  Synthetic
MYRMQLLSCIALSLALVTNSWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPSSSAGGGGSGGGGSGGGGSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSEQKLISEEDLHHHHHH

SEQ ID NO: 23 - protein, 20 aa, Human
MYRMQLLSCIALSLALVTNS

SEQ ID NO: 24 - protein, 18 aa, Artificial Sequence
  Synthetic
GSTSGSGKPGSGEGSTKG

SEQ ID NO: 25 - protein, 19 aa, Artificial Sequence
  Synthetic
SSSAGGGGSGGGGSGGGGS

SEQ ID NO: 26 - protein, 20 aa, Artificial Sequence
  Synthetic
SSSAGGGGSGGGGSGGGGSP

SEQ ID NO: 27 - protein, 10 aa, Human
EQKLISEEDL

SEQ ID NO: 28 - protein, 6 aa, Artificial Sequence
  Synthetic
HHHHHH

SEQ ID NO: 29 - protein, 201 aa, Murine
LDDAHSLRCNLTIKDPTPADPLWYEAKCLVDEILILHLSNINKTMTSGDPGETANATEVGECLTQPLKDLCQKLRNKVSNTKVDTHKTNGYPHLQVTMIYLQSQGQIPSATWEFNISDSYFFTFYTENMSWRSANDESGVIMNKWKDDGEFVKRLKFLIPECRQEVDEFLKQPKEKPRSTSRSPSITQLTSTSPLPPPSHS

SEQ ID NO: 30 - protein, 201 aa, Murine
LDDAHSLRCNLTIKDPTPADPLWYEAKCFVGEILILHLSNINKTMTSGDPGETANATEVKKCLTQPLKNLCQKLRNKVSNTKVDTHKTNGYPHLQVTMIYPQSQGRTPSATWEFNISDSYFFTFYTENMSWRSANDESGVIMNKWKDDGEFVKQLKFLIHECSQKMDEFLKQSKEKPRSTSRSPSITQLTSTSPLPPPSHS

SEQ ID NO: 31 - protein, 199 aa, Murine
LDDAHSLRCNLTIKAPTPADPLWYEAKCLVDEILILHLSNINKTMTSGDPGETANATEVGECLTQPVNDLCQKLRDKVSNTKVDTHKTNGYPHLQVTMIYPQSQGQTPSATWEFNISDSYFFTFYTENMSWRSANDESGVIMNKWNDDGDLVQRLKYFIPECRQKIDEFLKQSKEKPRSTSRSPSITQLTSTSPLPPPS

SEQ ID NO: 32 - protein, 197 aa, Murine
LDDAHSLRCNLTIKDPTSADLPWCDVKCSVDEITILHLNNINKTMTSGDPGKMANATGKCLTQPLNDLCQELRDKVSNTKVDTHKTNGYPHLQVTMIYPQSQGQTPSATWEFNISDSYFFTFYTENMSWRSANDESGVIMNKWKDDGDLVQQLKYFIPQCRQKIDEFLKQSKEKPRSTSRSPSITQLTSTSPLPPPS

SEQ ID NO: 33 - protein, 227 aa, Murine
TGTDSLSCELTFNHRTLHGQCSVNGKTLLDFGDKKHEGNATEMCADLSQSLRELSEGMRNQQSGNDALNVTTQSQYNQGEFIGGFWAINTDEQHSIYFYPLNMTWRESHSDNSSAMEHWKNKNLEKDIRNVLIIYFSRCLNKLSPHFREMPKSKIKVLDTTQNTNTTQIHPTVNNSQHNSDTQGLSFTWIVIICIGGIVSFMAFMVFAWCMLKKKKGALCCSSSSTT

SEQ ID NO: 34 - protein, 160 aa, Murine
LNCKLTVKYRTLQGLCSVNGKTFLDFGDENHEGNATMLCPALYQSLTDISEVMWSLQSGNDALNVTTRSQYYQGEFIDGFWDINTDEQHSIYVYPLNKTWRESHSDNSSAMEQWKNKNLEKDIRNVLMVDFSCCLNKSSPHFREMPTLPTTAAHVDQPRS